(19) United States
(12) Patent Application Publication
(10) Pub. No.: US 2004/0076985 A1
(43) Pub. Date: Apr. 22, 2004

(54) Regulation of human chemokine-like receptor

(76) Inventors: Alex Smolyar, Brookline, MA (US); Zhimin Zhu, Waban, MA (US); Jeffrey Encinas, Nara (JP); Shinichi Watanabe, Nara (JP); Hiromi Okigami, Kyoto (JP)

Correspondence Address: BANNER & WITCOFF, 1001 G STREET N W, SUITE 1100, WASHINGTON, DC, 20001, US

(21) Appl. No.: 10/450,590

(22) PCT Filed: Dec. 12, 2001
(86) PCT No.: PCT/EP01/14571

(51) Int. Cl. C12Q 1/68; G01N 33/53; C07H 21/04; C07K 14/715

(52) U.S. Cl. 435/6; 530/350; 435/7.1; 435/69.1; 435/320.1; 435/325; 536/23.5

(57) ABSTRACT

Reagents which regulate human chemokine-like receptor and reagents which bind to human chemokine-like receptor gene products can play a role in preventing, ameliorating, or correcting dysfunctions or diseases including, but not limited to, HIV infection, cardiovascular disorders, asthma and COPD.

This application incorporates by reference co-pending applications Serial No. 60/255,150 filed Dec. 14, 2000 and Serial No. 60/280,110 filed Apr. 2, 2001.

### TECHNICAL FIELD OF THE INVENTION

The invention relates to the area of receptor regulation. More particularly, the invention relates to the regulation of human chemokine-like receptor.

### BACKGROUND OF THE INVENTION

G Protein-Coupled Receptors

Many medically significant biological processes are mediated by signal transduction pathways that involve G-proteins (Lefkowitz, Nature 351, 353-354, 1991). The family of G protein-coupled receptors (GPCR) includes receptors for hormones, neurotransmitters, growth factors, and viruses. Specific examples of GPCRs include receptors for such diverse agents as calcitonin, adrenergic hormones, endothelin, cAMP, adenosine, acetylcholine, serotonin, dopamine, histamine, thrombin, kinin, follicle stimulating hormone, opsins, endothelial differentiation gene-1, rhodopsins, odorants, cytomegalovirus, G proteins themselves, effector proteins such as phos-pholipase C, adenyl cyclase, and phosphodiesterase, and actuator proteins such as protein kinase A and protein kinase C.

The GPCR protein superfamily now contains over 250 types of paralogues, receptors that represent variants generated by gene duplications (or other processes), as opposed to orthologues, the same receptor from different species. The superfamily can be broken down into five families: Family L receptors typified by rhodopsin and the β2-adrenergic receptor and currently represented by over 200 unique members (reviewed by Dohlman et al., Ann. Rev. Biochem. 60, 653-88, 1991, and references therein); Family II, the recently characterized parathyroid hormone/calcitonin/secretin receptor family (Juppner et al., Science 254, 1024-26, 1991; Lin et al., Science 254, 1022-24, 1991); Family III, the metabotropic glutamate receptor family in mammals (Nakanishi, Science 258, 597-603, 1992); Family IV, the cAMP receptor family, important in the chemotaxis and development of D. discoideum (Klein et al., Science 241, 1467-72, 1988; and Family V, the fingal mating pheromone receptors such as STE2 (reviewed by Kurjan, Ann. Rev. Biochem. 61, 1097-1129, 1992).

GPCRs possess seven conserved membrane-spanning domains connecting at least eight divergent hydrophilic loops. GPCRs (also known as 7TM receptors) have been characterized as including these seven conserved hydrophobic stretches of about 20 to 30 amino acids, connecting at least eight divergent hydrophilic loops. Most GPCRs have single conserved cysteine residues in each of the first two extracellular loops, which form disulfide bonds that are believed to stabilize functional protein structure. The seven transmembrane regions are designated as TM1, TM2, TM3, TM4, TM5, TM6, and TM7. TM3 has been implicated in signal transduction.

Phosphorylation and lipidation (palmitylation or farnesylation) of cysteine residues can influence signal transduction of some GPCRs. Most GPCRs contain potential phosphorylation sites within the third cytoplasmic loop and/or the carboxy terminus. For several GPCRs, such as the β-adrenergic receptor, phosphorylation by protein kinase A and/or specific receptor kinases mediates receptor desensitization.

For some receptors, the ligand binding sites of GPCRs are believed to comprise hydrophilic sockets formed by several GPCR transmembrane domains. The hydrophilic sockets are surrounded by hydrophobic residues of the GPCRs. The hydrophilic side of each GPCR transmembrane helix is postulated to face inward and form a polar ligand binding site. TM3 has been implicated in several GPCRs as having a ligand binding site, such as the TM3 aspartate residue. TM5 serines, a TM6 asparagine, and TM6 or TM7 phenylalanines or tyrosines also are implicated in ligand binding. GPCRs are coupled inside the cell by heterotrirneric G-proteins to various intra-cellular enzymes, ion channels, and transporters (see Johnson et al, Endoc. Rev. 10, 317-331, 1989). Different G-protein alpha-subunits preferentially stimulate particular effectors to modulate various biological functions in a cell. Phosphory-lation of cytoplasmic residues of GPCRs is an important mechanism for the regulation of some GPCRs. For example, in one form of signal transduction, the effect of hormone binding is the activation inside the cell of the enzyme, adenylate cyclase. Enzyme activation by hormones is dependent on the presence of the nucleotide GTP. GTP also influences hormone binding. A G protein connects the hormone receptor to adenylate cyclase. G protein exchanges GTP for bound GDP when activated by a hormone receptor. The GTP-carrying form then binds to activated adenylate cyclase. Hydrolysis of GTP to GDP, catalyzed by the G protein itself, returns the G protein to its basal, inactive form. Thus, the G protein serves a dual role, as an intermediate that relays the signal from receptor to effector, and as a clock that controls the duration of the signal.

Over the past 15 years, nearly 350 therapeutic agents targeting GPCRs receptors have been successfully introduced onto the market. This indicates that these receptors have an established, proven history as therapeutic targets. Clearly, there is an on-going need for identification and characterization of further GPCRs which can play a role in preventing, ameliorating, or correcting dysfunctions or diseases including, but not limited to, infections such as bacterial, fungal, protozoan, and viral infections, particularly those caused by HIV viruses, pain, cancers, anorexia, bulimia, asthma, Parkinson's diseases, acute heart failure, hypotension, hypertension, urinary retention, osteoporosis, angina pectoris, myocardial infarction, ulcers, asthma, allergies, benign prostatic hypertrophy, and psychotic and neurological disorders, including anxiety, schizophrenia, manic depression, delirium, dementia, several mental retardation, and dyskinesias, such as Huntington's disease and Tourett's syndrome.

Chemokine Receptors

Chemokines are a large family of low molecular weight, inducible, secreted, pro-inflammatory cytokines which are produced by various cell types. U.S. Pat. No. 5,955,303. They have been divided into several subfamilies on the basis of the positions of their conserved cysteines. The CXC family includes interleukin-8 (IL-8), growth regulatory gene, neutrophil-activating peptide-2, and platelet factor 4 (PF-4). Although IL-8 and PF-4 are both polymorphonuclear chemoattractants, angio-genesis is stimulated by IL-8 and inhibited by PF-4. The CC family includes monocyte chemoattractant protein-1 (MCP-1), RANTES (regulated on activation, normal T cell-expressed and secreted), macrophage inflammatory proteins (MIP-1.alpha., MIP-1.beta.), and eotaxin. MCP-1 is secreted by numerous cell types including endothelial, epithelial, and hematopoietic cells, and is a chemoattractant for monocytes and CD45RO+lymphocytes (Proost, P. (1996) Int J. Clin. Lab. Res. 26: 211-223; Raport, C. J. (1996) J. Biol. Chem. 271: 17161-17166).

Cells respond to chemokines through G-protein-coupled receptors. These receptors are seven transmembrane molecules which transduce their signal through hetero-trimeric GTP-binding proteins. Stimulation of the GTP-binding protein complex by activated receptor leads to the exchange of guanosine diphosphate for guanosine triphosphate and regulates the activity of effector molecules. There are distinct classes of each of the subunits which differ in activity and specificity and can elicit inhibitory or stimulatory responses. When stimulation of the known cytokine receptors shows agonist-dependent inhibition of adenylyl cyclase and mobilization of intracellular calcium, the receptor coupling to G_{α}i subunits (Myers, S. J. et al (1995) J. Biol. Chem. 270: 5786-5792).

Chemokine receptors play a major role in the mobilization and activation of cells of the immune system. The effects of receptor stimulation are dependent on the cell type and include chemotaxis, proliferation, differentiation, and production of cytokines. Chemokine stimulation produces changes in vascular endothelium, chemotaxis to sites of inflammation, and activates the effector functions of cells (Taub, D. D. (1996) Cytokine Growth Factor Rev. 7: 355-376).

The chemokine receptors display a range of sequence diversity and ligand promis-cuity. The known chemokine receptor protein sequence identities range from 22 to 40%, and certain receptors can respond to multiple ligands. Although mainly expressed in immune cells, viral homologues are expressed by human cytome-galovirus and Herpes virus saimiri. The chemokine receptor known as the Duffy blood group antigen binds both CC and CXC family chemokines and serves as the receptor on erythrocytes for the malarial parasite Plasmodium vivax. Chemokine receptors play a crucial role during the entry of human immunodefiency virus (HIV) into host cells. This initial event requires specific interactions between the viral envelope glycoprotein and two cellular receptors, CD4 and a chemokine coreceptor. The latter belongs to the family of seven-transmembrane G-protein-coupled receptors comprising the principal coreceptors CCR5, CXCR4 and others of minor importance including CCR3, CCR2b, CCR8, CX3CR1. Moore et al., Curr. Opin. Immunol 9, 551-562, 1997.

Chemokines appear to be involved in a variety of pro-inflammatory and autoimmune diseases, which makes them and their receptors very attractive therapeutic targets. In fact, small-molecule antagonists of seven of the chemokine receptor family have already been reported, some with potency in the low nanomolar range. Schwarz & Wells, Curr. Opin. Chem. Biol. 3, 407-17, 1999. It is likely that novel chemokines which affect the trafficking and activation of monocyte and CD8⁺ cells remain to be discovered.

Because of the importance of chemokine receptors, there is a need in the art to identify additional members of this receptor family that can be regulated to provide therapeutic effects.

### SUMMARY OF THE INVENTION

It is an object of the invention to provide reagents and methods of regulating a human chemokine-like receptor. This and other objects of the invention are provided by one or more of the embodiments described below.

One embodiment of the invention is a chemokine-like receptor polypeptide comprising an amino acid sequence selected from the group consisting of:

amino acid sequences which are at least about 26% identical to the amino acid sequence shown in SEQ ID NO: 2;

the amino acid sequence shown in SEQ ID NO: 2;

amino acid sequences which are at least about 26%% identical to the amino acid sequence shown in SEQ ID NO: 7;

the amino acid sequence shown in SEQ ID NO: 7;

amino acid sequences which are at least about 26% identical to the amino acid sequence shown in SEQ ID NO: 8 and

the amino acid sequence shown in SEQ ID NO: 8

Yet another embodiment of the invention is a method of screening for agents which decrease extracellular matrix degradation. A test compound is contacted with a chemokine-like receptor polypeptide comprising an amino acid sequence selected from the group consisting of:

amino acid sequences which are at least about 26% identical to the amino acid sequence shown in SEQ ID NO: 2;

the amino acid sequence shown in SEQ ID NO: 2;

amino acid sequences which are at least about 26%% identical to the amino acid sequence shown in SEQ ID NO: 7;

the amino acid sequence shown in SEQ ID NO: 7;

amino acid sequences which are at least about 26% identical to the amino acid sequence shown in SEQ ID NO: 8 and

the amino acid sequence shown in SEQ ID NO: 8

Binding between the test compound and the chemokine-like receptor polypeptide is detected. A test compound which binds to the chemokine-like receptor polypeptide is thereby identified as a potential agent for decreasing extracellular matrix degradation. The agent can work by decreasing the activity of the chemokine-like receptor.

Another embodiment of the invention is a method of screening for agents which decrease extracellular matrix degradation. A test compound is contacted with a polynucleotide encoding a chemokine-like receptor polypeptide, wherein the polynucleotide comprises a nucleotide sequence selected from the group consisting of:

nucleotide sequences which are at least about 50% identical to the nucleotide sequence shown in SEQ ID NO: 1;

the nucleotide sequence shown in SEQ ID NO: 1;

nucleotide sequences which are at least about 50% identical to the nucleotide sequence shown in SEQ ID NO: 4;

the nucleotide sequence shown in SEQ ID NO: 4;

nucleotide sequences which are at least about 50% identical to the nucleotide sequence shown in SEQ ID NO: 5;

the nucleotide sequence shown in SEQ ID NO: 5;

nucleotide sequences which are at least about 50% identical to the nucleotide sequence shown in SEQ ID NO: 9; and

the nucleotide sequence shown in SEQ ID NO: 9.

Binding of the test compound to the polynucleotide is detected. A test compound which binds to the polynucleotide is identified as a potential agent for decreasing extracellular matrix degradation. The agent can work by decreasing the amount of the chemokine-like receptor through interacting with the chemokine-like receptor mRNA.

Another embodiment of the invention is a method of screening for agents which regulate extracellular matrix degradation. A test compound is contacted with a chemokine-like receptor polypeptide comprising an amino acid sequence selected from the group consisting of:

amino acid sequences which are at least about 26% identical to the amino acid sequence shown in SEQ ID NO: 2;

the amino acid sequence shown in SEQ ID NO: 2;

amino acid sequences which are at least about 26%% identical to the amino acid sequence shown in SEQ ID NO: 7;

the amino acid sequence shown in SEQ ID NO: 7;

amino acid sequences which are at least about 26% identical to the amino acid sequence shown in SEQ ID NO: 8 and

the amino acid sequence shown in SEQ ID NO: 8

A chemokine-like receptor activity of the polypeptide is detected. A test compound which increases chemokine-like receptor activity of the polypeptide relative to chemokine-like receptor activity in the absence of the test compound is thereby identified as a potential agent for increasing extracellular matrix degradation. A test compound which decreases chemokine-like receptor activity of the polypeptide relative to chemokine-like receptor activity in the absence of the test compound is thereby identified as a potential agent for decreasing extracellular matrix degradation.

Even another embodiment of the invention is a method of screening for agents which decrease extracellular matrix degradation. A test compound is contacted with a chemokine-like receptor product of a polynucleotide which comprises a nucleotide sequence selected from the group consisting of:

nucleotide sequences which are at least about 50% identical to the nucleotide sequence shown in SEQ ID NO: 1;

the nucleotide sequence shown in SEQ ID NO: 1;

nucleotide sequences which are at least about 50% identical to the nucleotide sequence shown in SEQ ID NO: 4;

the nucleotide sequence shown in SEQ ID NO: 4;

nucleotide sequences which are at least about 50% identical to the nucleotide sequence shown in SEQ ID NO: 5;

the nucleotide sequence shown in SEQ ID NO: 5;

nucleotide sequences which are at least about 50% identical to the nucleotide sequence shown in SEQ ID NO: 9; and

the nucleotide sequence shown in SEQ ID NO: 9.

Binding of the test compound to the chemokine-like receptor product is detected. A test compound which binds to the chemokine-like receptor product is thereby identified as a potential agent for decreasing extracellular matrix degradation.

Still another embodiment of the invention is a method of reducing extracellular matrix degradation. A cell is contacted with a reagent which specifically binds to a polynucleotide encoding a chemokine-like receptor polypeptide or the product encoded by the polynucleotide, wherein the polynucleotide comprises a nucleotide sequence selected from the group consisting of:

nucleotide sequences which are at least about 50% identical to the nucleotide sequence shown in SEQ ID NO: 1;

the nucleotide sequence shown in SEQ ID NO: 1;

nucleotide sequences which are at least about 50% identical to the nucleotide sequence shown in SEQ ID NO: 4;

the nucleotide sequence shown in SEQ ID NO: 4;

nucleotide sequences which are at least about 50% identical to the nucleotide sequence shown in SEQ ID NO: 5;

the nucleotide sequence shown in SEQ ID NO: 5;

nucleotide sequences which are at least about 50% identical to the nucleotide sequence shown in SEQ ID NO: 9; and

the nucleotide sequence shown in SEQ ID NO: 9.

Chemokine-like receptor activity in the cell is thereby decreased.

The invention thus provides a human chemokine-like receptor which can be used to identify test compounds which may act, for example, as activators or inhibitors at the receptor's active site. Human chemokine-like receptor and fragments thereof also are useful in raising specific antibodies that can block the receptor and effectively reduce its activity.

### BRIEF DESCRIPTION OF THE DRAWINGS

FIG. 1 shows the DNA-sequence encoding a chemokine-like receptor Polypeptide (SEQ ID NO: 1).

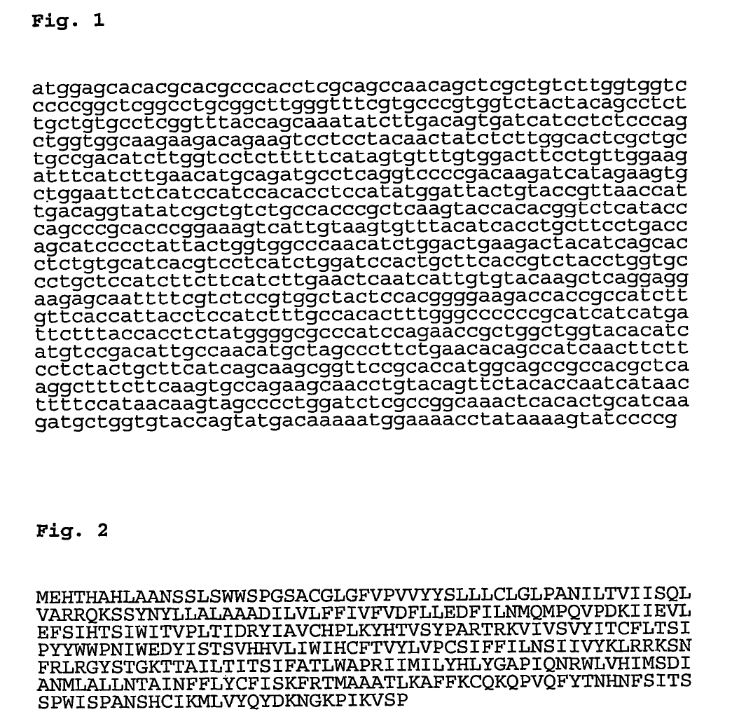

FIG. 2 shows the amino acid sequence deduced from the DNA-sequence of FIG. 1 (SEQ ID NO: 2).

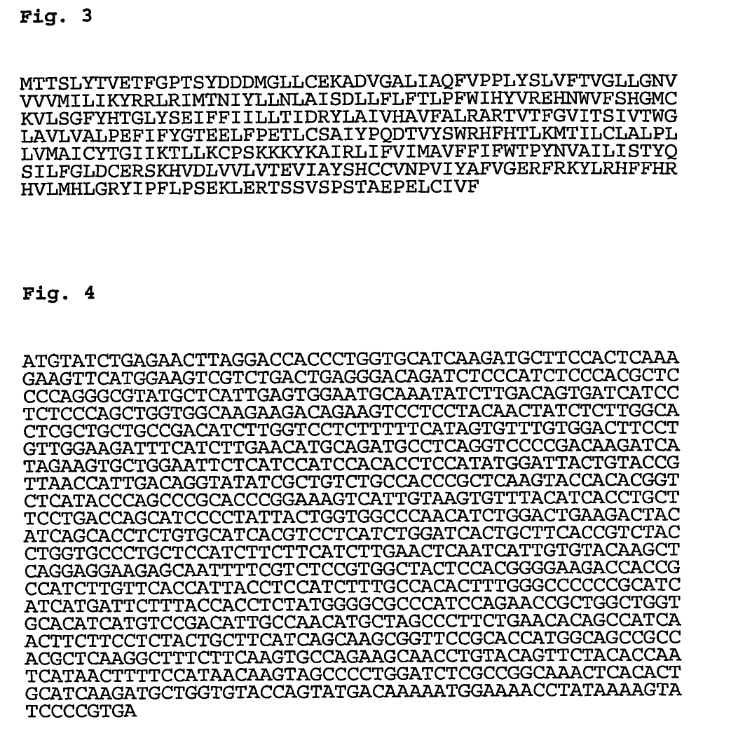

FIG. 3 shows the amino acid sequence of the protein identified by SwissProt Accession No. P56492 (SEQ ID NO: 3).

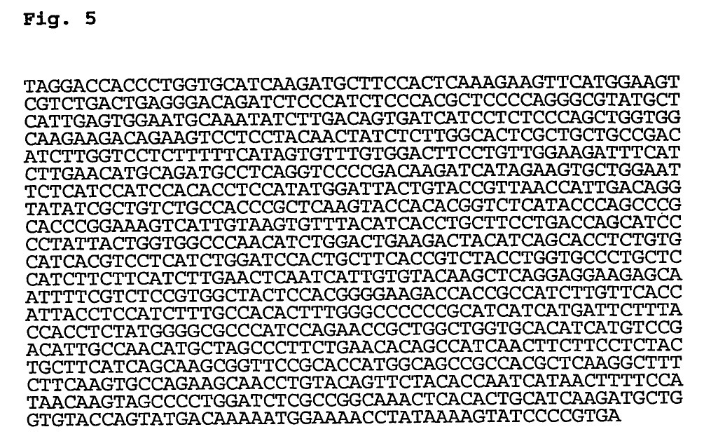

FIG. 4 shows the DNA-sequence encoding a chemokine-like receptor Polypeptide (SEQ ID NO: 4).

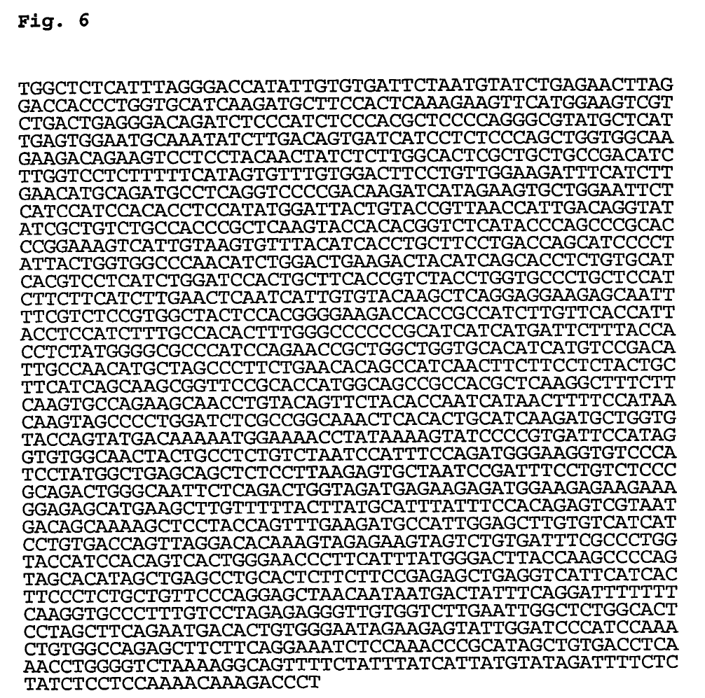

FIG. 5 shows the DNA-sequence encoding a chemokine-like receptor Polypeptide (SEQ ID NO: 5).

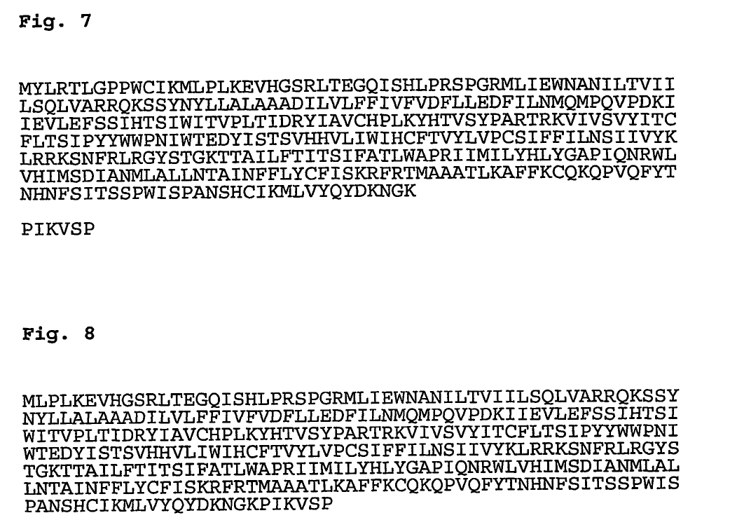

FIG. 6 shows the DNA-sequence encoding a chemokine-like receptor Polypeptide (SEQ ID NO: 6).

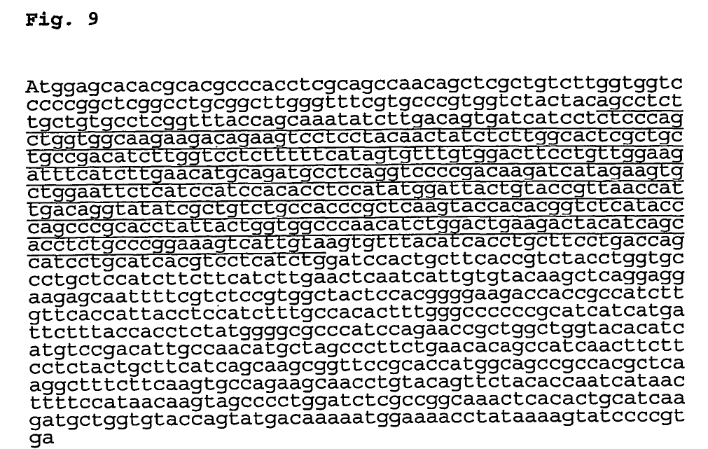

FIG. 7 shows the amino acid sequence deduced from the DNA-sequence of FIG. 4 (SEQ ID NO: 7).

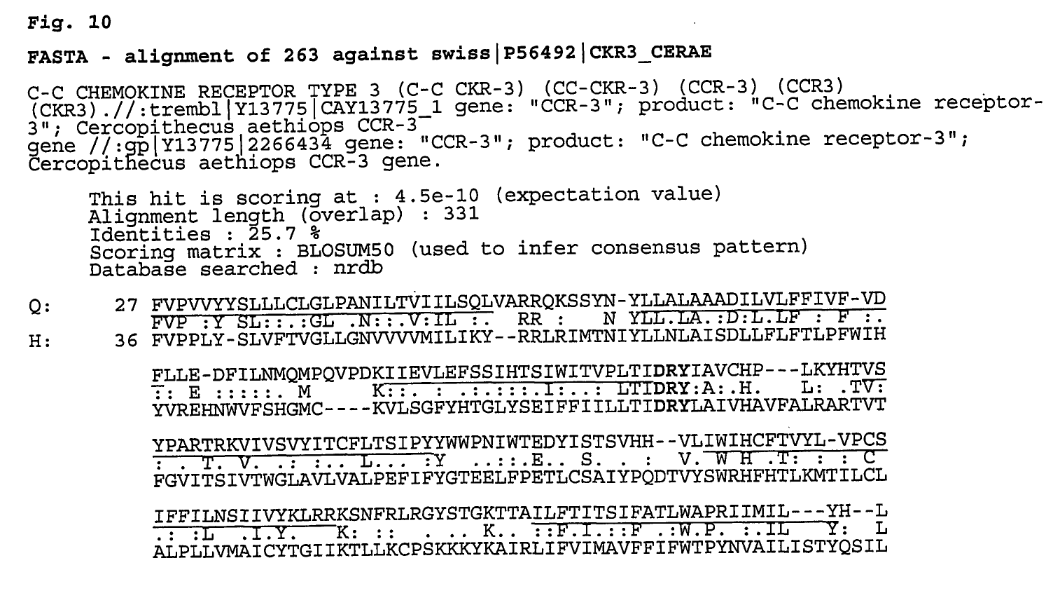

FIG. 8 shows the amino acid sequence deduced from the DNA-sequence of FIG. 5 (SEQ ID NO: 8).

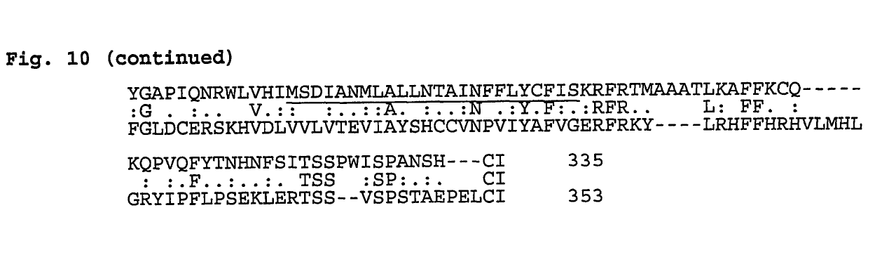

FIG. 9 shows the DNA-sequence encoding a chemokine-like receptor Polypeptide (SEQ ID NO: 9).

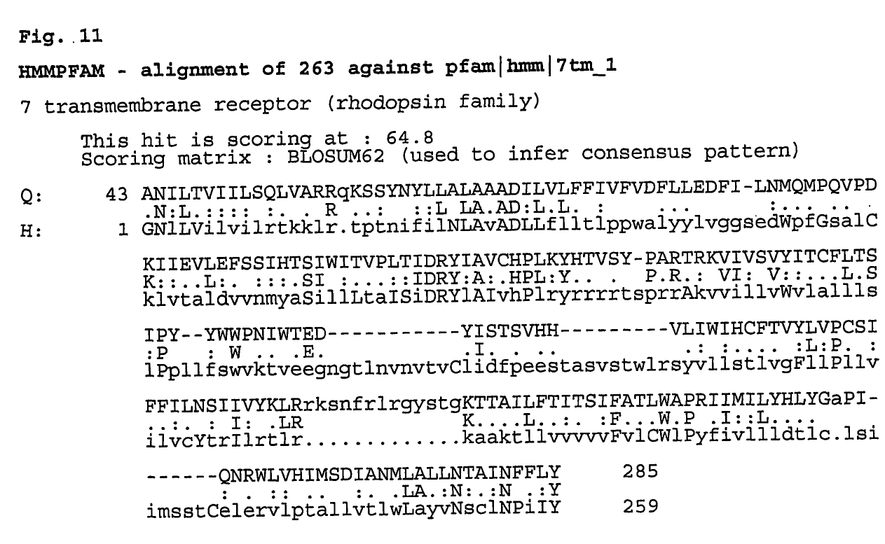

FIG. 10 shows the FASTA alignment of human chemokine-like receptor (SEQ ID NO: 2) with the protein identified with SwissProt Accession No. P56492 (SEQ ID NO: 3).

FIG. 11 shows the HMMPFAM alignment of SEQ ID NO: 2 against pfam|hmm|7tm_{—}1.

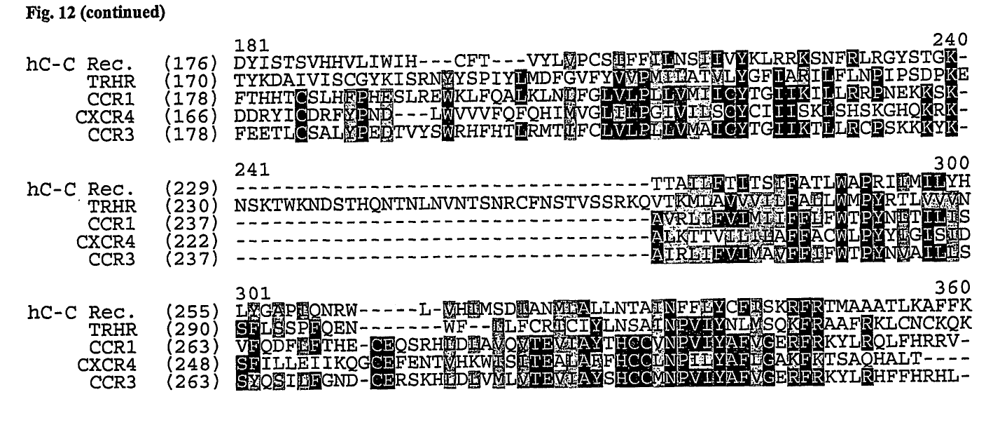

FIG. 12 shows alignment of the novel human chemokine receptor-like protein with its three closest human homologues, TRHR, CCR1, CXCR4, and CCR3. Dashes indicate where gaps were added to a sequence to improve the alignment. Background shading denotes the level of conservation between the five sequences at a particular residue, where black is identity between the five and decreasingly dark shades of gray show decreasing levels of conservation.

FIG. 13 shows the expression profiling of the novel human C-C chemokine receptor-like mRNA, whole-body screen.

FIG. 14 shows the expression profiling of the novel human C-C chemokine receptor-like mRNA, blood/lung screen.

### DETAILED DESCRIPTION OF THE INVENTION

The invention relates to an isolated polynucleotide encoding a chemokine-like receptor polypeptide and being selected from the group consisting of:

a) a polynucleotide encoding a chemokine-like receptor polypeptide comprising an amino acid sequence selected from the group consisting of:

amino acid sequences which are at least about 26% identical to the amino acid sequence shown in SEQ ID NO: 2; the amino acid sequence shown in SEQ ID NO: 2;

amino acid sequences which are at least about 26% identical to the amino acid sequence shown in SEQ ID NO: 7; the amino acid sequence shown in SEQ ID NO: 7;

amino acid sequences which are at least about 26% identical to the amino acid sequence shown in SEQ ID NO: 8; and

the amino acid sequence shown in SEQ ID NO: 8.

b) a polynucleotide comprising the sequence of SEQ ID NOS: 1, 4, 5 or 9;

c) a polynucleotide which hybridizes under stringent conditions to a poly-nucleotide specified in (a) and (b);

d) a polynucleotide the sequence of which deviates from the polynucleotide sequences specified in (a) to (c) due to the degeneration of the genetic code; and

e) a polynucleotide which represents a fragment, derivative or allelic variation of a polynucleotide sequence specified in (a) to (d).

Furthermore, it has been discovered by the present applicant that a novel chemokine-like receptor, particularly a human chemokine-like receptor, can be used in therapeutic methods to treat HV infection, cardiovascular disorders, asthma or COPD.

The novel human chemokine-like receptor transcript encodes a polypeptide of 356 amino acids with a calculated molecular mass of 41.4 kD. Analysis of the translation of human chemokine-like receptor reveals that the protein contains seven putative transmembrane domains, consistent with the structure of a GPCP. The gene is composed of two exons.

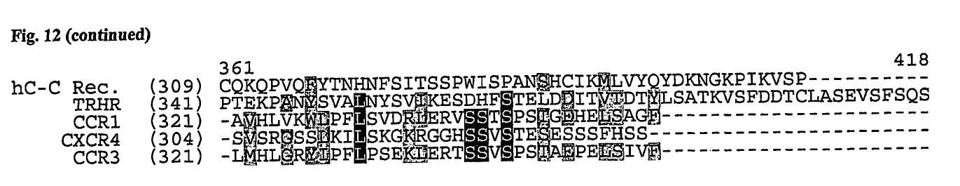

The amino acid sequence of human chemokine-like receptor shows 17.6% identity over its full length with its closest human homolog, C-C chemokine receptor 3 (CCR3). This value increases to an overall sequence similarity of 34.1% when amino acids with related physicochemical properties are included. Homology of human chemokine-like receptor with other chemokine receptors CCR1, CXCR1, and CXCR4 likewise shows an overall identity ranging from 13 to 17% and a similarity ranging from 29 to 32%. The novel human chemokine-like receptor additionally shows a similar degree of sequence homology to the thyrotropin releasing hormone receptor (TRHR), with an identity of 17.9% and a similarity of 32.8%, but structurally, TRHR has an extended third cytoplasmic loop between the fifth and sixth transmembrane domains that is not seen in human chemokine-like receptor (FIG. 12).

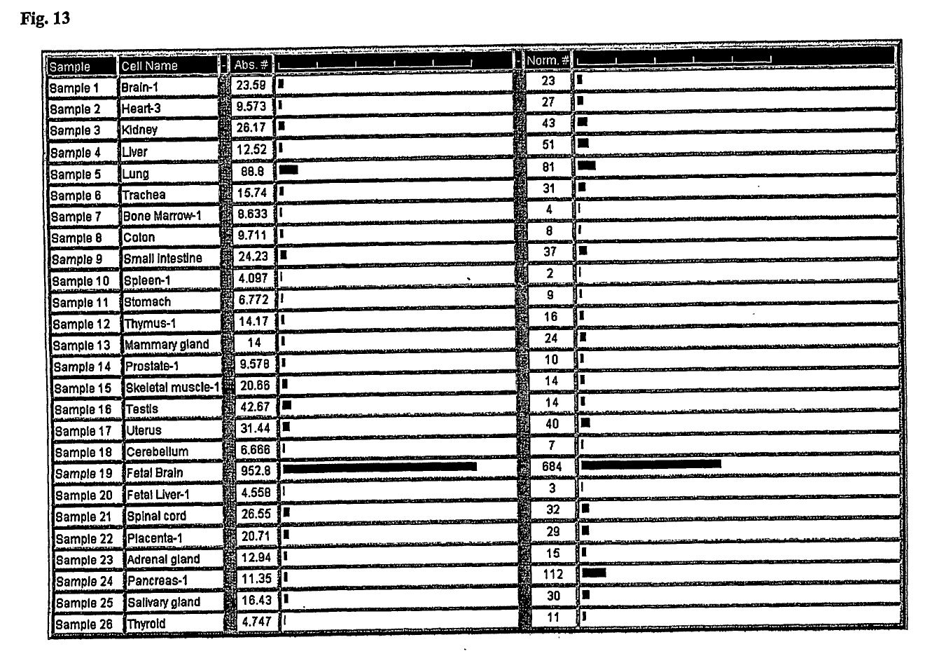

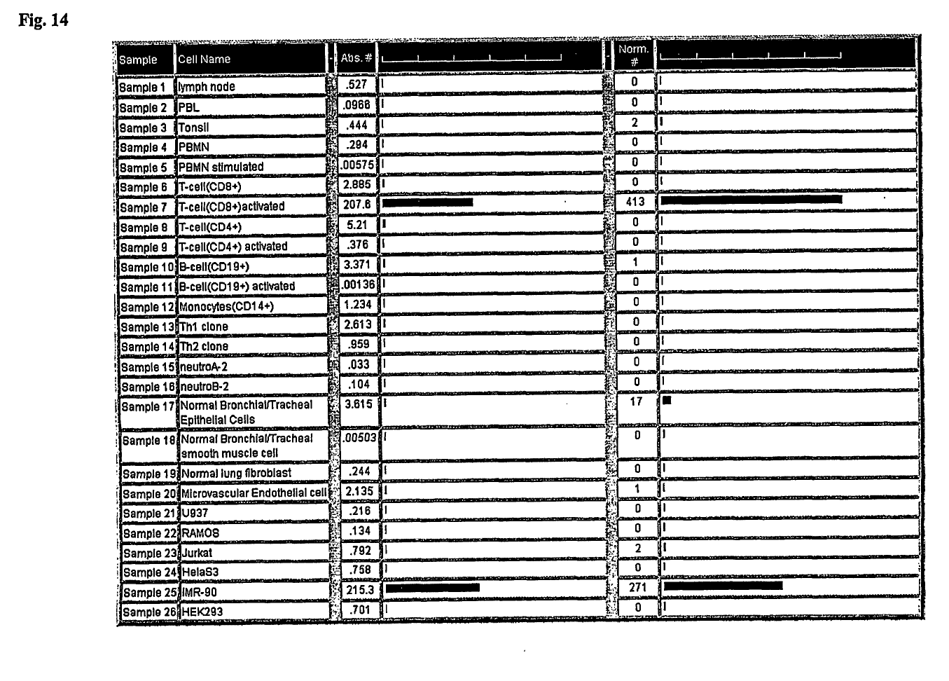

The distribution of human chemokine-like receptor mRNA expression was examined in several different human tissues, cell types, and commonly used cell lines (FIGS. 13 and 14). Among the tissues tested, fetal brain showed the most prominent expression, while pancreas and lung showed a moderate expression level. The novel human chemokine-like receptor appears to be expressed at low levels in most tissues, indicating expression on a cell type found in a variety of different tissues such as blood or vascular cells.

In specific cell types or cell lines tested, human chemokine-like receptor was found to be expressed at a high level in phytohemagglutinin-stimulated CD8⁺ cells, but strikingly in none of the other immune cells tested. High expression was also observed in a human fetal lung fibroblast line IMR-90, and moderate expression was seen in normal bronchial/tracheal epithelial cells.

Its high expression in activated CD8⁺ cells and its homology to chemokine receptors together suggest that the novel human chemokine-like receptor can act as a receptor of chemoattractant molecules on activated lymphocytes and thereby is involved, in a similar way to other chemokine receptors, in cell trafficking and homing to sites of infection, inflammation, or tissue injury. The regulation of activity of the novel human chemokine-like receptor therefore can be utilized to treat cardiovascular, immunological and inflammatory diseases, including but not limited to asthma and COPD. The combined expression in brain and CD8⁺ lymphocytes also suggests that this receptor is an advantageous target for viruses that reside in the nervous system. Therefore regulating the binding of ligands, for example chemoattractant molecules or virus particles, to this receptor can be used as a mechanism to modulate the immune response or to inhibit viral infections, including but not limited to HIV infection.

Human chemokine-like receptor comprises the amino acid sequence shown in SEQ ID NO: 2, 7, or 8. Coding sequences for human chemokine-like receptor are shown in SEQ ID NO: 1, 4, and 5. A longer sequence comprising the coding sequences is shown in SEQ ID NO: 5. This sequence is located on chromosome 16. Alternate start codons and the stop codon are shown in bold in FIG. 12.

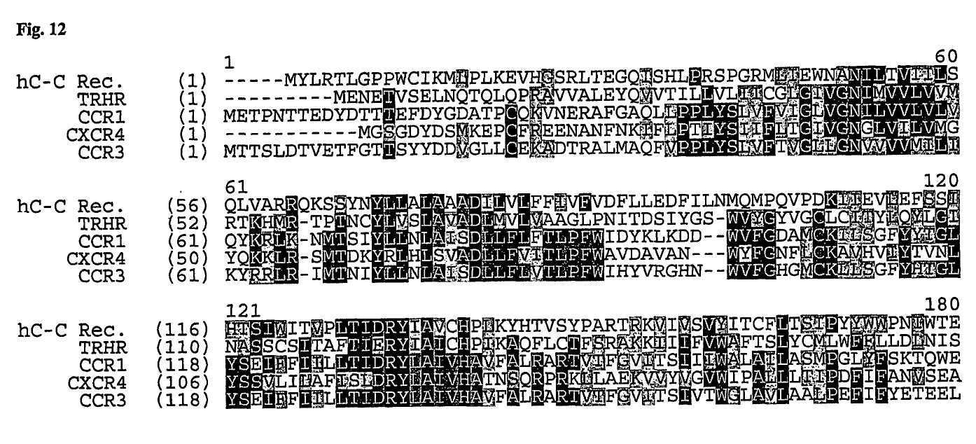

Human chemokine-like receptor is 24.7% identical over 331 amino acids to the protein identified with SwissProt Accession No. P56492 (SEQ ID NO: 3) and an-notated as “C-C CHEMOKINE RECEPTOR TYPE 3” (FIG. 10). Human chemokine-like receptor has a conserved acidic-Arg-aromatic triplet present in the second cytoplasmic loop, as shown in bold in FIG. 10.

Human chemokine-like receptor of the invention expected to be useful for the same purposes as previously identified chemokine receptors. Human chemokine-like receptor is believed to be useful in therapeutic methods to treat disorders such as HV infection, cardiovascular disorders, asthma and COPD. Human chemokine-like receptor also can be used to screen for human chemokine-like receptor activators and inhibitors.

Polypeptides

Human chemokine-like receptor polypeptides according to the invention comprise at least 6, 10, 15, 20, 25, 50, 75, 100, 125, 150, 175, 200, 225, 250, 275, 300, 325, 350, or 353 contiguous amino acids selected from the amino acid sequence shown in SEQ ID NO: 2, at least 6, 10, 15, 20, 25, 50, 75, 100, 125, 150, 175, 200, 225, 250, 275, 300, 325, 350, or 357 contiguous amino acids selected from the amino acid sequence shown in SEQ ID NO: 7, at least 6, 10, 15, 20, 25, 50, 75, 100, 125, 150, 175, 200, 225, 250, 275, 300, 325, or 344 contiguous amino acids selected from the amino acid sequence shown in SEQ ID NO: 8, or a biologically active variant thereof, as defined below. A chemokine-like receptor polypeptide of the invention therefore can be a portion of a chemokine-like receptor protein, a full-length chemokine-like receptor protein, or a fusion protein comprising all or a portion of a chemokine-like receptor protein.

Biologically Active Variants

Human chemokine-like receptor polypeptide variants that are biologically active, e.g., retain a chemokine activity, also are chemokine-like receptor polypeptides. Preferably, naturally or non-naturally occurring chemokine-like receptor polypeptide variants have amino acid sequences which are at least about 26, 30, 35, 40, 45, 50, 55, 60, 65, or 70, preferably about 75, 80, 85, 90, 96, 96, or 98% identical to the amino acid sequence shown in SEQ ID NO: 2, 7, or 8 or a fragment thereof. Percent identity between a putative chemokine-like receptor polypeptide variant and an amino acid sequence of SEQ ID NO: 2, 7, or 8 is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.).Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant. The FASTA algorithm is described y Pearson and Lipman, Proc. Nat'l Acad. Sci. USA 85:2444(1988), and by Pearson, Meth Enzymol. 183:63 (1990).Briefly, FASTA first characterizes sequence similarity by identifying regions shared by the query sequence (e.g. SEQ ID NO: 2, 7 or 8) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to for man approximate alignment with gaps. Finally, the highest scoring regions of the two amino acid sequences are aligned using a modification of the Needleman-Wunsch-Sellers algorithm (Needleman and Wunsch, J. Mol. Biol.48:444 (1970); Sellers, SIAM J. Appl. Math. 26:787 (1974)), which allows for amino acid insertions and deletions. Preferred parameters for FASTA analysis are: ktup=1, gapopeningpenalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).FASTA can also be used to determine the sequence identity of nucleic acid molecules using a ratio as disclosed above. For nucleotide sequence comparisons, the ktup value can range between one to six, preferably from three to six, most preferably three, with other parameters set as default.

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of a chemokine-like receptor polypeptide can be found using computer programs well known in the art, such as DNASTAR software. Whether an amino acid change results in a biologically active chemokine-like receptor polypeptide can readily be determined by assaying for chemokine receptor activity, as described for example, in U.S. Pat. No. 5,955,303.

Fusion Proteins

Fusion proteins are useful for generating antibodies against chemokine-like receptor polypeptide amino acid sequences and for use in various assay systems. For example, fusion proteins can be used to identify proteins which interact with portions of a chemokine-like receptor polypeptide. Protein affinity chromatography or library-based assays for protein-protein interactions, such as the yeast two-hybrid or phage display systems, can be used for this purpose. Such methods are well known in the art and also can be used as drug screens.

A chemokine-like receptor polypeptide fusion protein comprises two polypeptide segments fused together by means of a peptide bond. The first polypeptide segment comprises at least 6, 10, 15, 20, 25, 50, 75, 100, 125, 150, 175, 200, 225, 250, 275, 300, 325, 350, or 353 contiguous amino acids selected from the amino acid sequence shown in SEQ ID NO: 2, at least 6, 10, 15, 20, 25, 50, 75, 100, 125, 150, 175, 200, 225, 250, 275, 300, 325, 350, or 357 contiguous amino acids selected from the amino acid sequence shown in SEQ ID NO: 7, at least 6, 10, 15, 20, 25, 50, 75, 100, 125, 150, 175, 200, 225, 250, 275, 300, 325, or 344 contiguous amino acids selected from the amino acid sequence shown in SEQ ID NO: 8, or of a biologically active variant, such as those described above. The first polypeptide segment also can comprise full-length chemokine-like receptor protein.

The second polypeptide segment can be a full-length protein or a protein fragment. Proteins commonly used in fusion protein construction include β-galactosidase, β-glucuronidase, green fluorescent protein (GFP), autofluorescent proteins, including blue fluorescent protein (3BFP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein also can be engineered to contain a cleavage site located between the chemokine-like receptor polypeptide-encoding sequence and the heterologous protein sequence, so that the chemokine-like receptor polypeptide can be cleaved and purified away from the heterologous moiety.

A fusion protein can be synthesized chemically, as is known in the art. Preferably, a fusion protein is produced by covalently linking two polypeptide segments or by standard procedures in the art of molecular biology. Recombinant DNA methods can be used to prepare fusion proteins, for example, by making a DNA construct which comprises coding sequences selected from the complement of SEQ ID NO: 1 in proper reading frame with nucleotides encoding the second polypeptide segment and expressing the DNA construct in a host cell, as is known in the art Many kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada, 1-888-DNA-KITS).

Identification of Species Homologs

Species homologs of human chemokine-like receptor polypeptide can be obtained using chemokine-like receptor polypeptide polynucleotides (described below) to make suitable probes or primers for screening cDNA expression libraries from other species, such as mice, monkeys, or yeast, identifying cDNAs which encode homologs of chemokine-like receptor polypeptide, and expressing the cDNAs as is known in the art.

Polynucleotides

A chemokine-like receptor polynucleotide can be single- or double-stranded and comprises a coding sequence or the complement of a coding sequence for a chemokine-like receptor polypeptide. Coding sequences for human chemokine-like receptor are shown in SEQ ID NO: 1, 4, and 5.

Degenerate nucleotide sequences encoding human chemokine-like receptor polypeptides, as well as homologous nucleotide sequences which are at least about 50, 55, 60, 65, 70, preferably about 75, 90, 96, or 98% identical to the nucleotide sequence shown in SEQ ID NO: 1, 4, or 5 or its complement also are chemokine-like receptor polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of chemokine-like receptor polynucleotides which encode biologically active chemokine-like receptor polypeptides also are chemokine-like receptor polynucleotides.

Identification of Polynucleotide Variants and Homologs

Variants and homologs of the chemokine-like receptor polynucleotides described above also are chemokine-like receptor polynucleotides. Typically, homologous chemokine-like receptor polynucleotide sequences can be identified by hybridization of candidate polynucleotides to known chemokine-like receptor polynucleotides under stringent conditions, as is known in the art For example, using the following wash conditions—2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each—homologous sequences can be identified which contain at most about 25-30% basepair mismatches. More preferably, homologous nucleic acid strands contain 15-25% basepair mismatches, even more preferably 5-15% basepair mismatches.

Species homologs of the chemokine-like receptor polynucleotides disclosed herein also can be identified by making suitable probes or primers and screening cDNA expression libraries from other species, such as mice, monkeys, or yeast Human variants of chemokine-like receptor polynucleotides can be identified, for example, by screening human cDNA expression libraries. It is well known that the Tₘ of a double-stranded DNA decreases by 1-1.5° C. with every 1% decrease in homology (Bonner et al., J. Mol. Biol. 81, 123 (1973). Variants of human chemokine-like receptor polynucleotides or chemokine-like receptor polynucleotides of other species can therefore be identified by hybridizing a putative homologous chemokine-like receptor polynucleotide with a polynucleotide having a nucleotide sequence of SEQ ID NO: 1 or the complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to chemokine-like receptor polynucleotides or their complements following stringent hybridization and/or wash conditions also are chemokine-like receptor polynucleotides. Stringent wash conditions are well known and understood in the art and are disclosed, for example, in Sambrook et al., MOLECULAR CLONING: A LABORATORY MANUAL, 2d ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between a chemokine-like receptor polynucleotide having a nucleotide sequence shown in SEQ ID NO: 1, 4, or 5 or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tₘ=81.5° C.−16.6(log₁₀[Na⁺])+0.41(%G+C)−0.63(%formamide)−600/1),

where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

Preparation of Polynucleotides

A chemokine-like receptor polynucleotide can be isolated free of other cellular components such as membrane components, proteins, and lipids. Polynucleotides can be made by a cell and isolated using standard nucleic acid purification techniques, or synthesized using an amplification technique, such as the polymerase chain reaction (PCR), or by using an automatic synthesizer. Methods for isolating polynucleotides are routine and are known in the art. Any such technique for obtaining a polynucleotide can be used to obtain isolated chemokine-like receptor polynucleotides. For example, restriction receptors and probes can be used to isolate polynucleotide fragments which comprises chemokine-like nucleotide sequences. Isolated polynucleotides are in preparations which are free or at least 70, 80, or 90% free of other molecules.

Human chemokine-like receptor CDNA molecules can be made with standard molecular biology techniques, using chemokine-like receptor mRNA as a template. Human chemokine-like receptor cDNA molecules can thereafter be replicated using molecular biology techniques known in the art and disclosed in manuals such as Sambrook et al. (1989). An amplification technique, such as PCR, can be used to obtain additional copies of polynucleotides of the invention, using either human genomic DNA or cDNA as a template.

Alternatively, synthetic chemistry techniques can be used to synthesizes chemokine-like receptor polynucleotides. The degeneracy of the genetic code allows alternate nucleotide sequences to be synthesized which will encode a chemokine-like receptor polypeptide having, for example, an amino acid sequence shown in SEQ ID NO: 1 or a biologically active variant thereof.

Extending Polynucleotides

Various PCR-based methods can be used to extend the nucleic acid sequences disclosed herein to detect upstream sequences such as promoters and regulatory elements. For example, restriction-site PCR uses universal primers to retrieve unknown sequence adjacent to a known locus (Sarkar, PCR Methods Applic. 2, 318-322, 1993). Genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR also can be used to amplify or extend sequences using divergent primers based on a known region (Triglia et al., Nucleic Acids Res. 16, 8186, 1988). Primers can be designed using commercially available software, such as OLIGO 4.06 Primer Analysis software (National Biosciences Inc., Plymouth, Minn.), to be 22-30 nucleotides in length, to have a GC content of 50% or more, and to anneal to the target sequence at temperatures about 68-72° C. The method uses several restriction receptors to generate a suitable fragment in the known region of a gene. The fragment is then circularized by intramolecular ligation and used as a PCR template.

Another method which can be used is capture PCR, which involves PCR amplification of DNA fragments adjacent to a known sequence in human and yeast artificial chromosome DNA (Lagerstrom et al., PCR Methods Applic. 1, 111-119, 1991). In this method, multiple restriction receptor digestions and ligations also can be used to place an engineered double-stranded sequence into an unknown fragment of the DNA molecule before performing PCR.

Another method which can be used to retrieve unknown sequences is that of Parker et al., Nucleic Acids Res. 19, 3055-3060, 1991). Additionally, PCR, nested primers, and PROMOTERFINDER libraries (CLONTECH, Palo Alto, Calif.) can be used to walk genomic DNA (CLONTECH, Palo Alto, Calif.). This process avoids the need to screen libraries and is useful in finding intron/exon junctions.

When screening for full-length cDNAs, it is preferable to use libraries that have been size-selected to include larger cDNAs. Randomly-primed libraries are preferable, in that they will contain more sequences which contain the 5′ regions of genes. Use of a randomly primed library may be especially preferable for situations in which an oligo d(T) library does not yield a full-length cDNA. Genomic libraries can be useful for extension of sequence into 5′ non-transcribed regulatory regions.

Commercially available capillary electrophoresis systems can be used to analyze the size or confirm the nucleotide sequence of PCR or sequencing products. For example, capillary sequencing can employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate software (e.g. GENOTYPER and Sequence NAVIGATOR, Perkin Elmer), and the entire process from loading of samples to computer analysis and electronic data display can be computer controlled. Capillary electrophoresis is especially preferable for the sequencing of small pieces of DNA which might be present in limited amounts in a particular sample.

Obtaining Polypeptides

Human chemokine-like receptor polypeptides can be obtained, for example, by purification from human cells, by expression of chemokine-like receptor polynucleotides, or by direct chemical synthesis.

Protein Purification

Human chemokine-like receptor polypeptides can be purified from any cell which expresses the receptor, including host cells which have been transfected with chemokine-like receptor expression constructs. A purified chemokine-like receptor polypeptide is separated from other compounds which normally associate with the chemokine-like receptor polypeptide in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis. A preparation of purified chemokine-like receptor polypeptides is at least 80% pure; preferably, the preparations are 90%, 95%, or 99% pure. Purity of the preparations can be assessed by any means known in the art, such as SDS-polyacrylamide gel electrophoresis.

Expression of Polynucleotides

To express a chemokine-like receptor polynucleotide, the polynucleotide can be inserted into an expression vector which contains the necessary elements for the transcription and translation of the inserted coding sequence. Methods which are well known to those skilled in the art can be used to construct expression vectors containing sequences encoding chemokine-like receptor polypeptides and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., CURRENT PROTOCOLS IN MOLECULAR BIOLOGY, John Wiley & Sons, New York, N.Y., 1989.

A variety of expression vector/host systems can be utilized to contain and express sequences encoding a chemokine-like receptor polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems.

The control elements or regulatory sequences are those non-translated regions of the vector—enhancers, promoters, 5′ and 3′ untranslated regions—which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding a chemokine-like receptor polypeptide, vectors based on SV40 or EBV can be used with an appropriate selectable marker.

Bacterial and Yeast Expression Systems

In bacterial systems, a number of expression vectors can be selected depending upon the use intended for the chemokine-like receptor polypeptide. For example, when a large quantity of a chemokine-like receptor polypeptide is needed for the induction of antibodies, vectors which direct high level expression of fusion proteins that are readily purified can be used. Such vectors include, but are not limited to, multi-functional E. coli cloning and expression vectors such as BLUESCRIPT (Stratagene). In a BLUESCRIPT vector, a sequence encoding the chemokine-like receptor polypeptide can be ligated into the vector in frame with sequences for the amino-terminal Met and the subsequent 7 residues of β-galactosidase so that a hybrid protein is produced. pIN vectors (Van Heeke & Schuster, J. Biol. Chem. 264, 5503-5509, 1989) or pGEX vectors (Promega, Madison, Wis.) also can be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

In the yeast Saccharomyces cerevisiae, a number of vectors containing constitutive or inducible promoters such as alpha factor, alcohol oxidase, and PGH can be used. For reviews, see Ausubel et al. (1989) and Grant et al., Methods Enzymol. 153, 516-544, 1987.

Plant and Insect Expression Systems

If plant expression vectors are used, the expression of sequences encoding chemokine-like receptor polypeptides can be driven by any of a number of promoters. For example, viral promoters such as the 35S and 19S promoters of CaMV can be used alone or in combination with the omega leader sequence from TMV (Takamatsu, EMBO J. 6, 307-311, 1987). Alternatively, plant promoters such as the small subunit of RUBISCO or heat shock promoters can be used (Coruzzi et al., EMBO J. 3, 1671-1680, 1984; Broglie et al., Science 224, 838-843, 1984; Winter et al., Results Probl. Cell Differ. 17, 85-105, 1991). These constructs can be introduced into plant cells by direct DNA transformation or by pathogen-mediated transfection. Such techniques are described in a number of generally available reviews (e.g., Hobbs or Murray, in McGRAW HILL YEARBOOK OF SCIENCE AND TECHNOLOGY, McGraw Hill, New York, N.Y., pp. 191-196, 1992).

An insect system also can be used to express a chemokine-like receptor polypeptide. For example, in one such system Autographa californica nuclear polyhedrosis virus (AcNPV) is used as a vector to express foreign genes in Spodoptera frugiperda cells or in Trichoplusia larvae. Sequences encoding chemokine-like receptor polypeptides can be cloned into a non-essential region of the virus, such as the polyhedrin gene, and placed under control of the polyhedrin promoter. Successful insertion of chemokine-like receptor polypeptides will render the polyhedrin gene inactive and produce recombinant virus lacking coat protein. The recombinant viruses can then be used to infect S. frugiperda cells or Trichoplusia larvae in which chemokine-like receptor polypeptides can be expressed (Engelhard et al., Proc. Nat. Acad. Sci. 91, 3224-3227, 1994).

Mammalian Expression Systems

A number of viral-based expression systems can be used to express chemokine-like receptor polypeptides in mammalian host cells. For example, if an adenovirus is used as an expression vector, sequences encoding chemokine-like receptor polypeptides can be ligated into an adenovirus transcription/translation complex comprising the late promoter and tripartite leader sequence. Insertion in a non-essential E1 or E3 region of the viral genome can be used to obtain a viable virus which is capable of expressing a chemokine-like receptor polypeptide in infected host cells (Logan & Shenk, Proc. Natl. Acad. Sci. 81, 3655-3659, 1984). If desired, transcription enhancers, such as the Rous sarcoma virus (RSV) enhancer, can be used to increase expression in mammalian host cells.

Human artificial chromosomes (HACs) also can be used to deliver larger fragments of DNA than can be contained and expressed in a plasmid. HACs of 6M to 10M are constructed and delivered to cells via conventional delivery methods (e.g., liposomes, polycationic amino polymers, or vesicles).

Specific initiation signals also can be used to achieve more efficient translation of sequences encoding chemokine-like receptor polypeptides. Such signals include the ATG initiation codon and adjacent sequences. In cases where sequences encoding a chemokine-like receptor polypeptide, its initiation codon, and upstream sequences are inserted into the appropriate expression vector, no additional transcriptional or translational control signals may be needed. However, in cases where only coding sequence, or a fragment thereof, is inserted, exogenous translational control signals (including the ATG initiation codon) should be provided. The initiation codon should be in the correct reading frame to ensure translation of the entire insert. Exogenous translational elements and initiation codons can be of various origins, both natural and synthetic. The efficiency of expression can be enhanced by the inclusion of enhancers which are appropriate for the particular cell system which is used (see Scharf et al., Results Probl. Cell Differ. 20, 125-162, 1994).

Host Cells

A host cell strain can be chosen for its ability to modulate the expression of the inserted sequences or to process the expressed chemokine-like receptor polypeptide in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

Stable expression is preferred for long-term, high-yield production of recombinant proteins. For example, cell lines which stably express chemokine-like receptor polypeptides can be transformed using expression vectors which can contain viral origins of replication and/or endogenous expression elements and a selectable marker gene on the same or on a separate vector. Following the introduction of the vector, cells can be allowed to grow for 1-2 days in an enriched medium before they are switched to a selective medium. The purpose of the selectable marker is to confer resistance to selection, and its presence allows growth and recovery of cells which successfully express the introduced chemokine-like receptor sequences. Resistant clones of stably transformed cells can be proliferated using tissue culture techniques appropriate to the cell type. See, for example, ANIMAL CELL CULTURE, R. I. Freshney, ed., 1986.

Any number of selection systems can be used to recover transformed cell lines.

These include, but are not limited to, the herpes simplex virus thymidine kinase (Wigler et al., Cell 11, 223-32, 1977) and adenine phosphoribosyltransferase (Lowy et al., Cell 22, 817-23, 1980) genes which can be employed in tk⁻ or aprf cells, respectively. Also, antimetabolite, antibiotic, or herbicide resistance can be used as the basis for selection. For example, dhfr confers resistance to methotrexate (Wigler et al., Proc. Natl. Acad. Sci. 77, 3567-70, 1980), npt confers resistance to the aminoglycosides, neomycin and G-418 (Colbere-Garapin et al., J. Mol. Biol. 150, 1-14, 1981), and als and pat confer resistance to chlorsulfuron and phosphinotricin acetyltransferase, respectively (Murray, 1992, supra). Additional selectable genes have been described. For example, trpB allows cells to utilize indole in place of tryptophan, or hisD, which allows cells to utilize histinol in place of histidine (Hartman & Mulligan, Proc. Natl. Acad Sci. 85, 8047-51, 1988). Visible markers such as anthocyanins, β-glucuronidase and its substrate GUS, and luciferase and its substrate luciferin, can be used to identify transformants and to quantify the amount of transient or stable protein expression attributable to a specific vector system (Rhodes et al., Methods Mol. Biol. 55, 121-131, 1995).

Detecting Expression

Although the presence of marker gene expression suggests that the chemokine-like receptor polynucleotide is also present, its presence and expression may need to be confirmed. For example, if a sequence encoding a chemokine-like receptor polypeptide is inserted within a marker gene sequence, transformed cells containing sequences which encode a chemokine-like receptor polypeptide can be identified by the absence of marker gene function. Alternatively, a marker gene can be placed in tandem with a sequence encoding a chemokine-like receptor polypeptide under the control of a single promoter. Expression of the marker gene in response to induction or selection usually indicates expression of the chemokine-like receptor polynucleotide.

Alternatively, host cells which contain a chemokine-like receptor polynucleotide and which express a chemokine-like receptor polypeptide can be identified by a variety of procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a polynucleotide sequence encoding a chemokine-like receptor polypeptide can be detected by DNA-DNA or DNA-RNA hybridization or amplification using probes or fragments or fragments of polynucleotides encoding a chemokine-like receptor polypeptide. Nucleic acid amplification-based assays involve the use of oligonucleotides selected from sequences encoding a chemokine-like receptor polypeptide to detect transformants which contain a chemokine-like receptor polynucleotide.

A variety of protocols for detecting and measuring the expression of a chemokine-like receptor polypeptide, using either polyclonal or monoclonal antibodies specific for the polypeptide, are known in the art. Examples include receptor-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a chemokine-like receptor polypeptide can be used, or a competitive binding assay can be employed. These and other assays are described in Hampton et al., SEROLOGICAL METHODS: A LABORATORY MANUAL, APS Press, St. Paul, Minn., 1990) and Maddox et al., J. Exp. Med. 158,1211-1216,1983).

A wide variety of labels and conjugation techniques are known by those skilled in the art and can be used in various nucleic acid and amino acid assays. Means for producing labeled hybridization or PCR probes for detecting sequences related to polynucleotides encoding chemokine-like receptor polypeptides include oligo-labeling, nick translation, end-labeling, or PCR amplification using a labeled nucleotide. Alternatively, sequences encoding a chemokine-like receptor polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, receptors, and fluorescent, chemiluminescent, or chromogenic agents, as well as substrates, cofactors, inhibitors, magnetic particles, and the like.

Expression and Purification of Polypeptides

Host cells transformed with nucleotide sequences encoding a chemokine-like receptor polypeptide can be cultured under conditions suitable for the expression and recovery of the protein from cell culture. The polypeptide produced by a transformed cell can be secreted or contained intracellularly depending on the sequence and/or the vector used. As will be understood by those of skill in the art, expression vectors containing polynucleotides which encode chemokine-like receptor polypeptides can be designed to contain signal sequences which direct secretion of soluble chemokine-like receptor polypeptides through a prokaryotic or eukaryotic cell membrane or which direct the membrane insertion of membrane-bound chemokine-like receptor polypeptide.

As discussed above, other constructions can be used to join a sequence encoding a chemokine-like receptor polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Inclusion of cleavable linker sequences such as those specific for Factor Xa or enterokinase nitrogen, San Diego, Calif.) between the purification domain and the chemokine-like receptor polypeptide also can be used to facilitate purification. One such expression vector provides for expression of a fusion protein containing a chemokine-like receptor polypeptide and 6 histidine residues preceding a thioredoxin or an enterokinase cleavage site. The histidine residues facilitate purification by IMAC (immobilized metal ion affinity chromatography, as described in Porath et al., Prot. Exp. Purif. 3, 263-281, 1992), while the enterokinase cleavage site provides a means for purifying the chemokine-like receptor polypeptide from the fusion protein. Vectors which contain fusion proteins are disclosed in Kroll et al., DNA Cell Biol. 12, 441453, 1993.

Chemical Synthesis

Sequences encoding a chemokine-like receptor polypeptide can be synthesized, in whole or in part, using chemical methods well known in the art (see Caruthers et al., Nucl. Acids Res. Symp. Ser. 215-223, 1980; Horn et al. Nucl. Acids Res. Symp. Ser. 225-232, 1980). Alternatively, a chemokine-like receptor polypeptide itself can be produced using chemical methods to synthesize its amino acid sequence, such as by direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or by automation. Automated synthesis can be achieved, for example, using Applied Biosystems 431A Peptide Synthesizer (Perkin Elmer). Optionally, fragments of chemokine-like receptor polypeptides can be separately synthesized and combined using chemical methods to produce a full-length molecule.

The newly synthesized peptide can be substantially purified by preparative high performance liquid chromatography (e.g., Creighton, PROTEINS: STRUCRURES AND MOLECULAR PRINCIPLES, WH Freeman and Co., New York, N.Y., 1983). The composition of a synthetic chemokine-like receptor polypeptide can be confirmed by amino acid analysis or sequencing (e.g., the Edman degradation procedure; see Creighton, supra). Additionally, any portion of the amino acid sequence of the chemokine-like receptor polypeptide can be altered during direct synthesis and/or combined using chemical methods with sequences from other proteins to produce a variant polypeptide or a fusion protein.

Production of Altered Polyeptides

As will be understood by those of skill in the art, it may be advantageous to produce chemokine-like receptor polypeptide-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences disclosed herein can be engineered using methods generally known in the art to alter chemokine-like receptor polypeptide-encoding sequences for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

Antibodies

Any type of antibody known in the art can be generated to bind specifically to an epitope of a chemokine-like receptor polypeptide. “Antibody” as used herein includes intact immunoglobulin molecules, as well as fragments thereof, such as Fab, F(ab′)₂, and Fv, which are capable of binding an epitope of a chemokine-like receptor polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of a chemokine-like receptor polypeptide can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to a chemokine-like receptor polypeptide provides a detection signal at least 5-, 10-, or 20-fold higher than a detection signal provided with other proteins when used in an immunochemical assay. Preferably, antibodies which specifically bind to chemokine-like polypeptides do not detect other proteins in immunochemical assays and can immunoprecipitate a chemokine-like receptor polypeptide from solution.

Human chemokine-like receptor polypeptides can be used to immunize a mammal, such as a mouse, rat, rabbit, guinea pig, monkey, or human, to produce polyclonal antibodies. If desired, a chemokine-like receptor polypeptide can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a chemokine-like receptor polypeptide can be prepared using any technique which provides for the production of antibody molecules by continuous cell lines in culture. These techniques include, but are not limited to, the hybridoma technique, the human B-cell hybridoma technique, and the EBV-hybridoma technique (Kohler et al., Nature 256, 495497, 1985; Kozbor et al., J. Immunol. Methods 81, 3142, 1985; Cote et al., Proc. Natl. Acad. Sci. 80, 2026-2030, 1983; Cole et al., Mol. Cell Biol. 62, 109-120, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-6855, 1984; Neuberger et al., Nature 312, 604-608, 1984; Takeda et al., Nature 314, 452-454, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions. Alternatively, humanized antibodies can be produced using recombinant methods, as described in GB2188638B. Antibodies which specifically bind to a chemokine-like receptor polypeptide can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to chemokine-like receptor polypeptides. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., 1996, Eur. J. Cancer Prev. 5, 507-11). Single-chain antibodies can be mono- or bispecific, and can be bivalent or tetravalent Construction of tetravalent, bispecific single-chain antibodies is taught, for example, in Coloma & Morrison, 1997, Nat. Biotecinol. 15, 159-63. Construction of bivalent, bispecific single-chain antibodies is taught in Mallender & Voss, 1994, J. Biol. Chem. 269, 199-206.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., 1995, Int. J. Cancer 61, 497-501; Nicholls et al., 1993, J. Immunol. Meth. 165, 81-91).

Antibodies which specifically bind to chemokine-like receptor polypeptides also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833-3837, 1989; Winter et al., Nature 349, 293-299, 1991).

Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies according to the invention can be purified by methods well known in the art. For example, antibodies can be affinity purified by passage over a column to which a chemokine-like receptor polypeptide is bound. The bound antibodies can then be eluted from the column using a buffer with a high salt concentration.

Antisense Oligonucleotides

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of chemokine-like receptor gene products in the cell.

Antisense oligonucleotides can be deoxyribonucleotides, ribonucleotides, or a combination of both Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, Meth. Mol. Biol. 20, 1-8, 1994; Sonveaux, Meth. Mol. Biol. 26, 1-72, 1994; Uhlmann et al., Chem. Rev. 90, 543-583,1990.

Modifications of chemokine-like receptor gene expression can be obtained by designing antisense oligonucleotides which will form duplexes to the control, 5′, or regulatory regions of the chemokine-like receptor gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, MOLECULAR AND IMMUNOLOGIC APPROACHES, Futura Publishing Co., Mt. Kisco, N.Y., 1994). An antisense oligonucleotide also can be designed to block translation of mRNA by preventing the tanscript from binding to ribosomes.

Precise complementarity is not required for successful complex formation between an antisense oligonucleotide and the complementary sequence of a chemokine-like receptor polynucleotide. Antisense oligonucleotides which comprise, for example, 2, 3, 4, or 5 or more stretches of contiguous nucleotides which are precisely complementary to a chemokine-like receptor polynucleotide, each separated by a stretch of contiguous nucleotides which are not complementary to adjacent chemokine-like receptor nucleotides, can provide sufficient targeting specificity for chemokine-like receptor mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular chemokine-like receptor polynucleotide sequence.

Antisense oligonucleotides can be modified without affecting their ability to hybridize to a chemokine-like receptor polynucleotide. These modifications can be internal or at one or both ends of the antisense molecule. For example, inter-nucleoside phosphate linkages can be modified by adding cholesteryl or diamine moieties with varying numbers of carbon residues between the amino groups and terminal ribose. Modified bases and/or sugars, such as arabinose instead of ribose, or a 3′, 5′-substituted oligonucleotide in which the 3′ hydroxyl group or the 5′ phosphate group are substituted, also can be employed in a modified antisense oligonucleotide. These modified oligonucleotides can be prepared by methods well known in the art. See, e.g., Agrawal et al., Trends Biotechnol. 10, 152-158, 1992; Uhlmann et al., Chem. Rev. 90, 543-584, 1990; Uhlmann et al., Tetrahedron. Lett. 215, 3539-3542, 1987.

Ribozymes

Ribozymes are RNA molecules with catalytic activity. See, e.g., Cech, Science 236, 1532-1539; 1987; Cech, Ann. Rev. Biochem. 59, 543-568; 1990, Cech, Curr. Opin. Struct. Biol. 2, 605-609; 1992, Couture & Stinchcomb, Trends Genet. 12, 510-515, 1996. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of a chemokine-like receptor polynucleotide can be used to generate ribozymes which will specifically bind to mRNA transcribed from the chemokine-like receptor polynucleotide. Methods of designing and constructing ribozymes which can cleave other RNA molecules in trans in a highly sequence specific manner have been developed and described in the art (see Haseloff et al. Nature 334, 585-591, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201).

Specific ribozyme cleavage sites within a chemokine-like receptor RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate chemokine-like receptor RNA targets also can be evaluated by testing accessibility to hybridization with complementary oligonucleotides using ribonuclease protection assays. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease chemokine-like receptor expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal for controlling transcription of ribozymes in the cells.

As taught in Haseloff et al., U.S. Pat. No. 5,641,673, ribozymes can be engineered so that ribozyme expression will occur in response to factors which induce expression of a target gene. Ribozymes also can be engineered to provide an additional level of regulation, so that destruction of mRNA occurs only when both a ribozyme and a target gene are induced in the cells.

Differentially Expressed Genes

Described herein are methods for the identification of genes whose products interact with human chemokine-like receptor. Such genes may represent genes which are differentially expressed in disorders including, but not limited to, HIV infection, cardiovascular disorders, asthma and COPD. Further, such genes may represent genes which are differentially regulated in response to manipulations relevant to the progression or treatment of such diseases. Additionally, such genes may have a temporally modulated expression, increased or decreased at different stages of tissue or organism development. A differentially expressed gene may also have its expression modulated under control versus experimental conditions. In addition, the human chemokine-like receptor gene or gene product may itself be tested for differential expression.

The degree to which expression differs in a normal versus a diseased state need only be large enough to be visualized via standard characterization techniques such as differential display techniques. Other such standard characterization techniques by which expression differences may be visualized include but are not limited to, quantitative RT (reverse transcriptase), PCR, and Northern analysis.

Identification of Differentially Expressed Genes

To identify differentially expressed genes total RNA or, preferably, mRNA is isolated from tissues of interest For example, RNA samples are obtained from tissues of experimental subjects and from corresponding tissues of control subjects. Any RNA isolation technique which does not select against the isolation of mRNA may be utilized for the purification of such RNA samples. See, for example, Ausubel et al., ed.,, CURRENT PROTOCOLS IN MOLECULAR BIOLOGY, John Wiley & Sons, Inc. New York, 1987-1993. Large numbers of tissue samples may readily be processed using techniques well known to those of skill in the art, such as, for example, the single-step RNA isolation process of Chomczynski, U.S. Pat. No. 4,843,155.

Transcripts within the collected RNA samples which represent RNA produced by differentially expressed genes are identified by methods well known to those of skill in the art They include, for example, differential screening (Tedder et al., Proc. Natl. Acad. Sci. U.S.A. 85, 208-12, 1988), subtractive hybridization (Hedrick et al., Nature 308, 149-53; Lee et al., Proc. Natl. Acad Sci. U.S.A. 88, 2825, 1984), and, preferably, differential display (Liang & Pardee, Science 257, 967-71, 1992; U.S. Pat. No. 5,262,311).

The differential expression information may itself suggest relevant methods for the treatment of disorders involving the human chemokine-like receptor. For example, treatment may include a modulation of expression of the differentially expressed genes and/or the gene encoding the human chemokine-like receptor. The differential expression information may indicate whether the expression or activity of the differentially expressed gene or gene product or the human chemokine-like receptor gene or gene product are up-regulated or down-regulated.

Screening Methods

The invention provides assays for screening test compounds which bind to or modulate the activity of a chemokine-like receptor polypeptide or a chemokine-like receptor polynucleotide. A test compound preferably binds to a chemokine-like receptor polypeptide or polynucleotide. More preferably, a test compound decreases or increases chemokine-like by at least about 10, preferably about 50, more preferably about 75, 90, or 100% relative to the absence of the test compound.

Test Compounds

Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection The biological library approach is limited to polypeptide libraries, while the other four approaches are applicable to polypeptide, non-peptide oligomer, or small molecule libraries of compounds. See Lam, Anticancer Drug Des. 12, 145, 1997. Methods for the synthesis of molecular libraries are well known in the art (see, for example, DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, BioTechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. U.S.A. 89, 1865-1869, 1992), or phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol. Biol. 222, 301-310, 1991; and Ladner, U.S. Pat. No. 5,223,409).

High Throughput Screening

Test compounds can be screened for the ability to bind to chemokine-like receptor polypeptides or polynucleotides or to affect chemokine-like receptor activity or chemokine-like receptor gene expression using high throughput screening. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format.

Alternatively, “free format assays,” or assays that have no physical barrier between samples, can be used. For example, an assay using pigment cells (melanocytes) in a simple homogeneous assay for combinatorial peptide libraries is described by Jayawickreme et al., Proc. Natl. Acad. Sci. U.S.A. 19, 1614-18 (1994). The cells are placed under agarose in petri dishes, then beads that carry combinatorial compounds are placed on the surface of the agarose. The combinatorial compounds are partially released the compounds from the beads. Active compounds can be visualized as dark pigment areas because, as the compounds diffuse locally into the gel matrix, the active compounds cause the cells to change colors.

Another example of a free format assay is described by Chelsky, “Strategies for Screening Combinatorial Libraries: Novel and Traditional Approaches,” reported at the First Annual Conference of The Society for Biomolecular Screening in Philadelphia, Pa. (Nov. 7-10, 1995). Chelsky placed a simple homogenous receptor assay for carbonic anhydrase inside an agarose gel such that the receptor in the gel would cause a color change throughout the gel. Thereafter, beads carrying combinatorial compounds via a photolinker were placed inside the gel and the compounds were partially released by UV-light. Compounds that inhibited the receptor were observed as local zones of inhibition having less color change.

Yet another example is described by Salmon et al., Molecular Diversity 2, 57-63 (1996). In this example, combinatorial libraries were screened for compounds that had cytotoxic effects on cancer cells growing in agar.

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

Binding Assays

For binding assays, the test compound is preferably a small molecule which binds to and occupies, for example, the active site of the chemokine-like receptor polypeptide, such that normal biological activity is prevented Examples of such small molecules include, but are not limited to, small peptides or peptide-like molecules.

In binding assays, either the test compound or the chemokine-like receptor polypeptide can comprise a detectable label, such as a fluorescent, radioisotopic, chemiluminescent, or enzymatic label, such as horseradish peroxidase, alkaline phosphatase, or luciferase. Detection of a test compound which is bound to the chemokine-like receptor polypeptide can then be accomplished, for example, by direct counting of radioemmission, by scintillation counting, or by determining conversion of an appropriate substrate to a detectable product.

Alternatively, binding of a test compound to a chemokine-like receptor polypeptide can be determined without labeling either of the interactants. For example, a microphysiometer can be used to detect binding of a test compound with a chemokine-like receptor polypeptide. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between a test compound and a chemokine-like receptor polypeptide McConnell et al., Science 257, 1906-1912, 1992).

Determining the ability of a test compound to bind to a chemokine-like receptor polypeptide also can be accomplished using a technology such as real-time Bimolecular Interaction Analysis (BIA) (Sjolander & Urbaniczky, Anal. Chem. 63, 2338-2345, 1991, and Szabo et al., Curr. Opin. Struct. Biol. 5, 699-705, 1995). BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, a chemokine-like receptor polypeptide can be used as a “bait protein” in a two-hybrid assay or three-hybrid assay (see, e.g. U.S. Pat. No. 5,283,317; Zervos et al., Cell 72, 223-232, 1993; Madura et al., J. Biol. Chem. 268, 12046-12054, 1993; Bartel et al., BioTechniques 14, 920-924, 1993; Iwabuchi et al., Oncogene 8, 1693-1696, 1993; and Brent WO94/10300), to identify other proteins which bind to or interact with the chemokine-like receptor polypeptide and modulate its activity.

The two-hybrid system is based on the modular nature of most transcription factors, which consist of separable DNA-binding and activation domains. Briefly, the assay utilizes two different DNA constructs. For example, in one construct, polynucleotide encoding a chemokine-like receptor polypeptide can be fused to a polynucleotide encoding the DNA binding domain of a known transcription factor (e.g., GAL-4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein which interacts with the chemokine-like receptor polypeptide.

It may be desirable to immobilize either the chemokine-like receptor polypeptide (or polynucleotide) or the test compound to facilitate separation of bound from unbound forms of one or both of the interactants, as well as to accommodate automation of the assay. Thus, either the chemokine-like receptor polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach the receptor polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked. Binding of a test compound to a chemokine-like receptor polypeptide (or polynucleotide) can be accomplished in any vessel suitable for containing the reactants. Examples of such vessels include microtiter plates, test tubes, and microcentrifuge tubes.

In one embodiment, the chemokine-like receptor polypeptide is a fusion protein comprising a domain that allows the chemokine-like receptor polypeptide to be bound to a solid support. For example, glutathione-S-transferase fusion proteins can be adsorbed onto glutathione sepharose beads (Sigma Chemical, St. Louis, Mo.) or glutathione derivatized microtiter plates, which are then combined with the test compound or the test compound and the non-adsorbed chemokine-like receptor polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, either a chemokine-like receptor polypeptide (or polynucleotide) or a test compound can be immobilized utilizing conjugation of biotin and streptavidin. Biotinylated chemokine-like receptor polypeptides (or polynucleotides) or test compounds can be prepared from biotin-NHS(N-hydroxysuccinimide) using techniques well known in the art (e.g., biotinylation kit, Pierce Chemicals, Rockford, Ill.) and immobilized in the wells of streptavidin-coated 96 well plates (Pierce Chemical). Alternatively, antibodies which specifically bind to a chemokine-like receptor polypeptide, polynucleotide, or a test compound, but which do not interfere with a desired binding site, such as the active site of the chemokine-like receptor polypeptide, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to the chemokine-like receptor polypeptide or test compound, receptor-linked assays which rely on detecting an activity of the chemokine-like receptor polypeptide, and SDS gel electrophoresis under non-reducing conditions.

Screening for test compounds which bind to a chemokine-like receptor polypeptide or polynucleotide also can be carried out in an intact cell. Any cell which comprises a chemokine-like receptor polypeptide or polynucleotide can be used in a cell-based assay system. A chemokine-like receptor polynucleotide can be naturally occurring in the cell or can be introduced using techniques such as those described above. Binding of the test compound to a chemokine-like receptor polypeptide or polynucleotide is determined as described above.

Functional Assays

Test compounds can be tested for the ability to increase or decrease a biological effect of an chemokine polypeptide. Such biological effects can be determined using the functional assays described in the specific examples, below. Functional assays can be carried out after contacting either a purified polypeptide, a cell membrane preparation, or an intact cell with a test compound. A test compound which decreases a functional activity of an chemokine by at least about 10, preferably about 50, more preferably about 75, 90, or 100% is identified as a potential agent for decreasing chemokine. A t st compound which increases chemokine activity by at least about 10, preferably about 50, more preferably about 75, 90, or 100% is identified as a potential agent for increasing chemokine.

Gene Expression

In another embodiment, test compounds which increase or decrease chemokine-like receptor gene expression are identified. A chemokine-like receptor polynucleotide is contacted with a test compound, and the expression of an RNA or polypeptide product of the chemokine-like receptor polynucleotide is determined. The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of chemokine-like receptor mRNA or polypeptide expression in the cells can be determined by methods well known in the art for detecting mRNA or polypeptide. Either qualitative or quantitative methods can be used. The presence of polypeptide products of a chemokine-like receptor polynucleotide can be determined, for example, using a variety of techniques known in the art, including immunochemical methods such as radioimmunoassay, Western blotting, and immuno-histochemistry. Alternatively, polypeptide synthesis can be determined in vivo, in a cell culture, or in an in vitro translation system by detecting incorporation of labeled amino acids into a chemokine-like receptor polypeptide.

Such screening can be carried out either in a cell-free assay system or in an intact cell. Any cell which expresses a chemokine-like receptor polynucleotide can be used in a cell-based assay system. The chemokine-like receptor polynucleotide can be naturally occurring in the cell or can be introduced using techniques such as those described above. Either a primary culture or an established cell line, such as CHO or human embryonic kidney 293 cells, can be used.

Pharmaceutical Compositions

The invention also provides pharmaceutical compositions which can be administered to a patient to achieve a therapeutic effect. Pharmaceutical compositions of the invention can comprise, for example, a chemokine-like receptor polypeptide, chemokine-like receptor polynucleotide, ribozymes or antisense oligonucleotides, antibodies which specifically bind to a chemokine-like receptor polypeptide, or mimetics, agonists, antagonists, or inhibitors of a chemokine-like receptor polypeptide activity. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically-acceptable carriers comprising excipients and auxiliaries which facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers. Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the 5 suspension also can contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. in other cases, the preferred preparation can be a lyophilized powder which can contain any or all of the following: 1-50 mM histidine, 0.1%-2% sucrose, and 2-7% mannitol, at a pH range of 4.5 to 5.5, that is combined with buffer prior to use.

Further details on techniques for formulation and administration can be found in the latest edition of REMINGTON'S PHARMACEUTICAL SCIENCES (Maack Publishing Co., Easton, Pa.). After pharmaceutical compositions have been prepared, they can be placed in an appropriate container and labeled for treatment of an indicated condition. Such labeling would include amount, frequency, and method of administration.

Therapeutic Indications and Methods

Human chemokine-like receptor can be regulated to treat HIV infection, cardiovascular diseases, asthma and COPD.

Cardiovascular diseases include the following disorders of the heart and the vascular system: congestive heart failure, myocardial infarction, ischemic diseases of the heart, all kinds of atrial and ventricular arrhythmias, hypertensive vascular diseases, and peripheral vascular diseases.

Heart failure is defined as a pathophysiologic state in which an abnormality of cardiac function is responsible for the failure of the heart to pump blood at a rate commensurate with the requirement of the metabolizing tissue. It includes all forms of pumping failure, such as high-output and low-output, acute and chronic, right-sided or left-sided, systolic or diastolic, independent of the underlying cause.

Myocardial infarction (MI) is generally caused by an abrupt decrease in coronary blood flow that follows a thrombotic occlusion of a coronary artery previously narrowed by arteriosclerosis. MI prophylaxis (primary and secondary prevention) is included, as well as the acute treatment of MI and the prevention of complications.

Ischemic diseases are conditions in which the coronary flow is restricted resulting in a perfusion which is inadequate to meet the myocardial requirement for oxygen. This group of diseases includes stable angina, unstable angina, and asymptomatic ischemia.

Arrhythmias include all forms of atrial and ventricular tachyarrhythmias (atrial tachycardia, atrial flutter, atrial fibrillation, atrio-ventricular reentrant tachycardia, preexcitation syndrome, ventricular tachycardia, ventricular flutter, and ventricular fibrillation), as well as bradycardic forms of arrhythmias.

Vascular diseases include primary as well as all kinds of secondary arterial hypertension (renal, endocrine, neurogenic, others). The disclosed gene and its product may be used as drug targets for the treatment of hypertension as well as for the prevention of all complications. Peripheral vascular diseases are defined as vascular diseases in which arterial and/or venous flow is reduced resulting in an imbalance between blood supply and tissue oxygen demand. It includes chronic peripheral arterial occlusive disease (PAOD), acute arterial thrombosis and embolism, inflammatory vascular disorders, Raynaud's phenomenon, and venous disorders.

Allergy is a complex process in which environmental antigens induce clinically adverse reactions. The inducing antigens, called allergens, typically elicit a specific IgE response and, although in most cases the allergens themselves have little or no intrinsic toxicity, they induce pathology when the IgE response in turn elicits an IgE-dependent or T cell-dependent hypersensitivity reaction. Hypersensitivity reactions can be local or systemic and typically occur within minutes of allergen exposure in individuals who have previously been sensitized to an allergen. The hypersensitivity reaction of allergy develops when the allergen is recognized by IgE antibodies bound to specific receptors on the surface of effector cells, such as mast cells, basophils, or eosinophils, which causes the activation of the effector cells and the release of mediators that produce the acute signs and symptoms of the reactions. Allergic diseases include asthma, allergic rhinitis (hay fever), atopic dermatitis, and anaphylaxis.

Asthma is though to arise as a result of interactions between multiple genetic and environmental factors and is characterized by three major features: 1) intermittent and reversible airway obstruction caused by bronchoconstriction, increased mucus production, and thickening of the walls of the airways that leads to a narrowing of the airways, 2) airway hyperresponsiveness caused by a decreased control of airway caliber, and 3) airway inflammation. Certain cells are critical to the inflammatory reaction of asthma and they include T cells and antigen presenting cells, B cells that produce IgE, and mast cells, basophils, eosinophils, and other cells that bind IgE. These effector cells accumulate at the site of allergic reaction in the airways and release toxic products that contribute to the acute pathology and eventually to the tissue destruction related to the disorder. Other resident cells, such as smooth muscle cells, lung epithelial cells, mucus-producing cells, and nerve cells may also be abnormal in individuals with asthma and may contribute to the pathology. While the airway obstruction of asthma, presenting clinically as an intermittent wheeze and shortness of breath, is generally the most pressing symptom of the disease requiring immediate treatment, the inflammation and tissue destruction associated with the disease can lead to irreversible changes that eventually make asthma a chronic disabling disorder requiring long-term management.

Despite recent important advances in our understanding of the pathophysiology of asthma, the disease appears to be increasing in prevalence and severity (Gergen and Weiss, Am. Rev. Respir. Dis. 146, 823-24, 1992). It is estimated that 3040% of the population suffer with atopic allergy, and 15% of children and 5% of adults in the population suffer from asthma (Gergen and Weiss, 1992). Thus, an enormous burden is placed on our health care resources. However, both diagnosis and treatment of asthma are difficult. The severity of lung tissue inflammation is not easy to measure and the symptoms of the disease are often indistinguishable from those of respiratory infections, chronic respiratory inflammatory disorders, allergic rhinitis, or other respiratory disorders. Often, the inciting allergen cannot be determined, making removal of the causative environmental agent difficult. Current pharmacological treatments suffer their own set of disadvantages. Commonly used therapeutic agents, such as beta activators, can act as symptom relievers to transiently improve pulmonary function, but do not affect the underlying inflammation. Agents that can reduce the underlying inflammation, such as anti-inflammatory steroids, can have major drawbacks that range from immunosuppression to bone loss (Goodman and Gilman's THE PHARMACOLOGIC BASIS OF THERAPEUTICS, Seventh Edition, MacMillan Publishing Company, NY, USA, 1985). In addition, many of the present therapies, such as inhaled corticosteroids, are short-lasting, inconvenient to use, and must be used often on a regular basis, in some cases for life, making failure of patients to comply with the treatment a major problem and thereby reducing their effectiveness as a treatment.

Because of the problems associated with conventional therapies, alternative treatment strategies have been evaluated. Glycophorin A (Chu and Sharom, Cell. Immunol. 145, 223-39, 1992), cyclosporin (Alexander et al., Lancet 339, 324-28, 1992), and a nonapeptide fragment of IL-2 (Zav'yalov et al., Immunol. Lett. 31, 285-88, 1992) all inhibit interleukin-2 dependent T lymphocyte proliferation; however, they are known to have many other effects. For example, cyclosporin is used as a immunosup-pressant after organ transplantation. While these agents may represent alternatives to steroids in the treatment of asthmatics, they inhibit interleukin-2 dependent T lymphocyte proliferation and potentially critical immune functions associated with homeostasis. Other treatments that block the release or activity of mediators of bronchochonstriction, such as cromones or anti-leukotrienes, have recently been introduced for the treatment of mild asthma, but they are expensive and not effective in all patients and it is unclear whether they have any effect on the chronic changes associated with asthmatic inflammation. What is needed in the art is the identification of a treatment that can act in pathways critical to the development of asthma_that both blocks the episodic attacks of the disorder and preferentially dampens the hyperactive allergic immune response without immunocompromising the patient.

Chronic obstructive pulmonary (or airways) disease (COPD) is a condition defined physiologically as airflow obstruction that generally results from a mixture of emphysema and peripheral airway obstruction due to chronic bronchitis (Senior & Shapiro, Pulmonary Diseases and Disorders, 3d ed., New York, McGraw-Hill, 1998, pp. 659-681, 1998; Barnes, Chest 117, 10S-14S, 2000). Emphysema is characterized by destruction of alveolar walls leading to abnormal enlargement of the air spaces of the lung. Chronic bronchitis is defined clinically as the presence of chronic productive cough for three months in each of two successive years. In COPD, airflow obstruction is usually progressive and is only partially reversible. By far the most important risk factor for development of COPD is cigarette smoking, although the disease does occur in non-smokers.

Chronic inflammation of the airways is a key pathological feature of COPD (Senior & Shapiro, 1998). The inflammatory cell population comprises increased numbers of macrophages, neutrophils, and CD8⁺ lymphocytes. Inhaled irritants, such as cigarette smoke, activate macrophages which are resident in the respiratory tract, as well as epithelial cells leading to release of chemokines (e.g. interleukin-8) and other chemotactic factors. These chemotactic factors act to increase the neutrophil/-monocyte trafficking from the blood into the lung tissue and airways. Neutrophils and monocytes recruited into the airways can release a variety of potentially damaging mediators such as proteolytic enzymes and reactive oxygen species. Matrix degradation and emphysema, along with airway wall thickening, surfactant dysfunction, and mucus hypersecretion, all are potential sequelae of this inflammatory response that lead to impaired airflow and gas exchange.

Several GPCRs have been implicated in the pathology of COPD. For example, the chemokine IL-8 acts through CXCR1 and CXCR2, and antagonists for these receptors are under investigation as therapeutics for COPD. Members of the P2Y family of metabotropic receptors may play key roles in normal pulmonary function. In particular, the P2Y₂ receptor is believed to be involved in the regulation of mucociliary clearance mechanisms in the lung, and agonists of this receptor may stimulate airway mucus clearance in patients with chronic bronchitis (Yerxa Johnson, Drugs of the Future 24, 759-769, 1999). GPCRs, therefore, are therapeutic targets for COPD, and the identification of additional members of existing GPCR families or of novel GPCRs would yield further attractive targets.

This invention further pertains to the use of novel agents identified by the screening assays described above. Accordingly, it is within the scope of this invention to use a test compound identified as described herein in an appropriate animal model. For example, an agent identified as described herein (e.g., a modulating agent, an antisense nucleic acid molecule, a specific antibody, ribozyme, or a chemokine-like receptor polypeptide binding molecule) can be used in an animal model to determine the efficacy, toxicity, or side effects of treatment with such an agent Alternatively, an agent identified as described herein can be used in an animal model to determine the mechanism of action of such an agent. Furthermore, this invention pertains to uses of novel agents identified by the above-described screening assays for treatments as described herein.

A reagent which affects chemokine-like receptor activity can be administered to a human cell, either in vitro or in vivo, to reduce chemokine-like receptor activity. The reagent preferably binds to an expression product of a human chemokine-like receptor gene. If the expression product is a protein, the reagent is preferably an antibody. For treatment of human cells ex vivo, an antibody can be added to a preparation of stem cells which have been removed from the body. The cells can then be replaced in the same or another human body, with or without clonal propagation, as is known in the art.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to a specific organ of an animal, such as the lung, liver, spleen, heart brain, lymph nodes, and skin.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 10⁶ cells, more preferably about 1.0 μg of DNA per 16 nmole of liposome delivered to about 10⁶ cells, and even more preferably about 2.0 μg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes standardly used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome.

Complexing a liposome with a reagent such as an antisense oligonucleotide or ribozyme can be achieved using methods which are standard in the art (see, for example, U.S. Pat. No. 5,705,151). Preferably, from about 0.1 μg to about 10 μg of polynucleotide is combined with about 8 nmol of liposomes, more preferably from about 0.5 μg to about 5 μg of polynucleotides are combined with about 8 nmol liposomes, and even more preferably about 1.0 μg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al. Trends in Biotechnol. 11, 202-05 (1993); Chiou et al., GENE THERAPEUTICS: METHODS AND APPLICATIONS OF DIRECT GENE TRANSFER (J. A. Wolff, ed.) (1994); Wu & Wu, J. Biol. Chem. 263, 621-24 (1988); Wu et al., J. Biol. Chem. 269, 542-46 (1994); Zenke et al., Proc. Natl. Acad. Sci. U.S.A. 87, 3655-59 (1990); Wu et al., J. Biol. Chem. 266, 338-42 (1991).

Determination of α Therapeutically Effective Dose

The determination of a therapeutically effective dose is well within the capability of those skilled in the art. A therapeutically effective dose refers to that amount of active ingredient which increases or decreases chemokine-like receptor activity relative to the chemokine-like receptor activity which occurs in the absence of the therapeutically effective dose.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

If the reagent is a single-chain antibody, polynucleotides encoding the antibody can be constructed and introduced into a cell either ex vivo or in vivo using well-established techniques including, but not limited to, tansferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

Effective in vivo dosages of an antibody are in the range of about 5 μg to about 50 μg/kg, about 50 μg to about 5 mg/kg, about 100 μg to about 500 μg /kg of patient body weight, and about 200 to about 250 μg/kg of patient body weight. For administration of polynucleotides encoding single-chain antibodies, effective in vivo dosages are in the range of about 100 μg to about 200 μg, 500 μg to about 50 mg, about 1 μg to about 2 mg, about 5 μg to about 500 μg, and about 20 μg to about 100 μg of DNA.

If the expression product is mRNA, the reagent is preferably an antisense oligonucleotide or a ribozyme. Polynucleotides which express antisense oligonucleotides or ribozymes can be introduced into cells by a variety of methods, as described above.

Preferably, a reagent reduces expression of a chemokine-like receptor gene or the activity of a chemokine-like receptor polypeptide by at least about 10, preferably about 50, more preferably about 75, 90, or 100% relative to the absence of the reagent The effectiveness of the mechanism chosen to decrease the level of expression of a chemokine-like receptor gene or the activity of a chemokine-like receptor polypeptide can be assessed using methods well known in the art, such as hybridization of nucleotide probes to chemokine-like receptor-specific mRNA, quantitative RT-PCR, immunologic detection of a chemokine-like receptor polypeptide, or measurement of chemokine-like receptor activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Any of the therapeutic methods described above can be applied to any subject in need of such therapy, including, for example, mammals such as dogs, cats, cows, horses, rabbits, monkeys, and most preferably, humans.

Diagnostic Methods

Human chemokine-like receptor also can be used in diagnostic assays for detecting diseases and abnormalities or susceptibility to diseases and abnormalities related to the presence of mutations in the nucleic acid sequences which encode the receptor. For example, differences can be determined between the cDNA or genomic sequence encoding chemokine-like receptor in individuals afflicted with a disease and in normal individuals. If a mutation is observed in some or all of the afflicted individuals but not in normal individuals, then the mutation is likely to be the causative agent of the disease.

Sequence differences between a reference gene and a gene having mutations can be revealed by the direct DNA sequencing method. In addition, cloned DNA segments can be employed as probes to detect specific DNA segments. The sensitivity of this method is greatly enhanced when combined with PCR. For example, a sequencing primer can be used with a double-stranded PCR product or a single-stranded template molecule generated by a modified PCR. The sequence determination is performed by conventional procedures using radiolabeled nucleotides or by automatic sequencing procedures using fluorescent tags.

Genetic testing based on DNA sequence differences can be carried out by detection of alteration in electrophoretic mobility of DNA fragments in gels with or without denaturing agents. Small sequence deletions and insertions can be visualized, for example, by high resolution gel electrophoresis. DNA fragments of different sequences can be distinguished on denaturing formamide gradient gels in which the mobilities of different DNA fragments are retarded in the gel at different positions according to their specific melting or partial melting temperatures (see, e.g., Myers et al., Science 230, 1242, 1985). Sequence changes at specific locations can also be revealed by nuclease protection assays, such as RNase and S 1 protection or the chemical cleavage method (e.g., Cotton et al., Proc. Natl Acad. Sci. USA 85, 4397-4401, 1985). Thus, the detection of a specific DNA sequence can be performed by methods such as hybridization, RNase protection, chemical cleavage, direct DNA sequencing or the use of restriction receptors and Southern blotting of genomic DNA. In addition to direct methods such as gel-electrophoresis and DNA sequencing, mutations can also be detected by in situ analysis.

Altered levels of a chemokine-like receptor also can be detected in various tissues. Assays used to detect levels of the receptor polypeptides in a body sample, such as blood or a tissue biopsy, derived from a host are well known to those of skill in the art and include radioimmunoassays, competitive binding assays, Western blot analysis, and ELISA assays.

All patents and patent applications cited in this disclosure are expressly incorporated herein by reference. The above disclosure generally describes the present invention. A more complete understanding can be obtained by reference to the following specific examples which are provided for purposes of illustration only and are not intended to limit the scope of the invention.

### EXAMPLE 1

Detection of Chemokine-like Receptor Activity

The polynucleotide of SEQ D NO: 1 is inserted into the expression vector pCEV4 and the expression vector pCEV4-chemokine-like receptor polypeptide obtained is transfected into human embryonic kidney 293 cells. From these cells extracts are obtained and centrifuged at 1000 rpm for 5 minutes at 4° C. The supernatant is centrifuged at 30,000×g for 20 minutes at 4° C. The pellet is suspended in binding buffer containing 50 mM Tris HCl, 5 mM MgSO₄, 1 mM EDTA, 100 mM NaCl, pH 7.5, supplemented with 0.1% BSA, 2 μg/ml aprotinin, 0.5 mg/ml leupeptin, and 10 μg/ml phosphoramidon. Optimal membrane suspension dilutions, defined as the protein concentration required to bind less than 10% of the added radioligand, i.e. chemokine, are added to 96-well polypropylene microtiter plates containing ¹²⁵I-labeled ligand, non-labeled peptides, and binding buffer to a final volume of 250 μl.

In equilibrium saturation binding assays, membrane preparations are incubated in the presence of increasing concentrations (0.1 nM to 4 nM) of ¹²⁵I-labeled ligand.

Binding reaction mixtures are incubated for one hour at 30° C. The reaction is stopped by filtration through GF/B filters treated with 0.5% polyethyleneimine, using a cell harvester. Radioactivity is measured by scintillation counting, and data are analyzed by a computerized non-linear regression program.

Non-specific binding is defined as the amount of radioactivity remaining after incubation of membrane protein in the presence of 100 nM of unlabeled peptide. Protein concentration is measured by the Bradford method using Bio-Rad Reagent, with bovine serum albumin as a standard. It is shown that the polypeptide of SEQ ID NO: 2 has a chemokine-like receptor activity.

### EXAMPLE 2

Expression of Recombinant Human Chemokine-like Receptor

The Pichia pastoris expression vector pPICZB (Invitrogen, San Diego, Calif.) is used to produce large quantities of recombinant human chemokine-like polypeptides in yeast. The chemokine-like receptor-encoding DNA sequence is derived from SEQ ID NO: 1, 4, or 5. Before insertion into vector pPICZB, the DNA sequence is modified by well known methods in such a way that it contains at its 5′-end an initiation codon and at its 3′-end an enterokinase cleavage site, a His6 reporter tag and a termination codon. Moreover, at both termini recognition sequences for restriction endonucleases are added and after digestion of the multiple cloning site of pPICZB with the corresponding restriction receptors the modified DNA sequence is ligated into pPICZB. This expression vector is designed for inducible expression in Pichia pastoris, driven by a yeast promoter. The resulting pPICZ/md-His6 vector is used to transform the yeast.

The yeast is cultivated under usual conditions in 5 liter shake flasks and the recombinantly produced protein isolated from the culture by affinity chromatography (Ni-NTA-Resin) in the presence of 8 M urea. The bound polypeptide is eluted with buffer, pH 3.5, and neutralized. Separation of the polypeptide from the His6 reporter tag is accomplished by site-specific proteolysis using enterokinase (Invitrogen, San Diego, Calif.) according to manufacturer's instructions. Purified human chemokine-like receptor polypeptide is obtained.

### EXAMPLE 3

Identification of Test Compounds that Bind to Chemokine-like Receptor Polypeptides

Purified chemokine-like receptor polypeptides comprising a glutathione-S-transferase protein and absorbed onto glutathione-derivatized wells of 96-well microtiter plates are contacted with test compounds from a small molecule library at pH 7.0 in a physiological buffer solution. Human chemokine-like receptor polypeptides comprise the amino acid sequence shown in SEQ ID NO: 2, 7, or 8. The test compounds comprise a fluorescent tag. The samples are incubated for 5 minutes to one hour. Control samples are incubated in the absence of a test compound.

The buffer solution containing the test compounds is washed from the wells. Binding of a test compound to a chemokine-like receptor polypeptide is detected by fluorescence measurements of the contents of the wells. A test compound which increases the fluorescence in a well by at least 15% relative to fluorescence of a well in which a test compound is not incubated is identified as a compound which binds to a chemokine-like receptor polypeptide.

### EXAMPLE 4

Identification of a Test Compound which Decreases Chemokine-like Receptor Gene Expression

A test compound is administered to a culture of human cells transfected with a chemokine-like receptor expression construct and incubated at 37° C. for 10 to 45 minutes. A culture of the same type of cells which have not been transfected is incubated for the same time without the test compound to provide a negative control.

RNA is isolated from the two cultures as described in Chirgwin et al., Biochem. 18, 5294-99, 1979). Northern blots are prepared using 20 to 30 μg total RNA and hybridized with a ³²P-labeled chemokine-like receptor-specific probe at 65° C. in Express-hyb (CLONTECH). The probe comprises at least 11 contiguous nucleotides selected from the complement of SEQ ID NO: 1. A test compound which decreases the chemokine-like receptor-specific signal relative to the signal obtained in the absence of the test compound is identified as an inhibitor of chemokine-like receptor gene expression.

### EXAMPLE 5

Identification of a Test Compound which Decreases Chemokine-like Receptor Activity

A test compound is administered to a culture of human cells transfected with a chemokine-like receptor expression construct and incubated at 37° C. for 10 to 45 minutes. A culture of the same type of cells which have not been transfected is incubated for the same time without the test compound to provide a negative control. Chemokine receptor activity is measured using the method of U.S. Pat. No. 5,955,303.

A test compound which decreases the chemokine activity of the chemokine-like receptor relative to the chemokine activity in the absence of the test compound is identified as an inhibitor of chemokine-like receptor activity.

### EXAMPLE 6

Tissue-specific Expression of Chemokine Receptor-like Protein

To demonstrate that chemokine receptor-like protein is involved in the disease process of COPD, the initial expression panel consists of RNA samples from respiratory tissues and inflammatory cells relevant to COPD: lung (adult and fetal), trachea, freshly isolated alveolar type II cells, cultured human bronchial epithelial cells, cultured small airway epithelial cells, cultured bronchial sooth muscle cells, cultured H441 cells (Clara-like), freshly isolated neutrophils and monocytes, and cultured monocytes (macrophage-like). Body map profiling also is carried out, using total RNA panels purchased from Clontech. The tissues are adrenal gland, bone marrow, brain, colon, heart, kidney, liver, lung, mammary gland, pancreas, prostate, salivary gland, skeletal muscle, small intestine, spleen, stomach, testis, thymus, trachea, thyroid, and uterus.

Quantitative expression profiling. Quantitative expression profiling is performed by the form of quantitative PCR analysis called “kinetic analysis” firstly described in Higuchi et al., BioTechnology 10, 413-17, 1992, and Higuchi et al., BioTechnology 11, 1026-30, 1993. The principle is that at any given cycle within the exponential phase of PCR, the amount of product is proportional to the initial number of template copies.

If the amplification is performed in the presence of an internally quenched fluorescent oligonucleotide (TaqMan probe) complementary to the target sequence, the probe is cleaved by the 5′-3′ endonuclease activity of Taq DNA polymerase and a fluorescent dye released in the medium (Holland et al., Proc. Natl. Acad. Sci. U.S.A. 88, 7276-80, 1991). Because the fluorescence emission will increase in direct proportion to the amount of the specific amplified product, the exponential growth phase of PCR product can be detected and used to determine the initial template concentration (Heid et al., Genome Res. 6, 986-94, 1996, and Gibson et al., Genome Res. 6, 995-1001, 1996).

The amplification of an endogenous control can be performed to standardize the amount of sample RNA added to a reaction. In this kind of experiment, the control of choice is the 18S ribosomal RNA. Because reporter dyes with differing emission spectra are available, the target and the endogenous control can be independently quantified in the same tube if probes labeled with different dyes are used. All “real time PCR” measurements of fluorescence are made in the ABI Prism 7700. RNA extraction and cDNA preparation. Total RNA from the tissues listed above are used for expression quantification. RNAs labeled “from autopsy” were extracted from autoptic tissues with the TRIzol reagent (Life Technologies, MD) according to the manufacturer's protocol.

Fifty μg of each RNA are treated with DNase I for 1 hour at 37° C. in the following reaction mix: 0.2 U/μl RNase-free DNase I (Roche Diagnostics, Germany); 0.4 U/μl RNase inhibitor (PE Applied Biosystems, CA); 10 mM Tris-HCl pH 7.9; 10 mM MgCl₂; 50 mM NaCl; and 1 mM DTT.

After incubation, RNA is extracted once with 1 volume of phenol:chloroform:-isoamyl alcohol (24:24:1) and once with chloroform, and precipitated with 1/10 volume of 3 M NaAcetate, pH5.2, and 2 volumes of ethanol.

Fifty μg of each RNA from the autoptic tissues are DNase treated with the DNA-free kit purchased from Ambion (Ambion, Tex.). After resuspension and spectrophotometric quantification, each sample is reverse transcribed with the TaqMan Reverse Transcription Reagents (PE Applied Biosystems, CA) according to the manufacturer's protocol. The final concentration of RNA in the reaction mix is 200 ng/μL. Reverse transcription is carried out with 2.5 μM of random hexamer primers. TaqMan quantitative analysis. Specific primers and probe are designed according to the recommendations of PE Applied Biosystems. Probes are labeled either FAM=6-carboxy-fluorescein or with TAMRA=6-carboxy-tetratnethyl-rhodamine. Quantification experiments are performed on 10 ng of reverse transcribed RNA from each sample. Each determination is done in triplicate.

Total cDNA content is normalized with the simultaneous quantification (multiplex PCR) of the 18S ribosomal RNA using the Pre-Developed TaqMan Assay Reagents (PDAR) Control Kit (PE Applied Biosystems, CA).

The assay reaction mix is as follows: 1×final TaqMan Universal PCR Master Mix (from 2×stock) (PE Applied Biosystems, CA); 1×PDAR control—18S RNA (from 20×stock); 300 nM forward primer, 900 nM reverse primer; 200 nM probe; 10 ng cDNA; and water to 25 μl.

Each of the following steps are carried out once: pre PCR, 2 minutes at 50° C., and 10 minutes at 95° C. The following steps are carried out 40 times: denaturation, 15 seconds at 95° C., annealing/extension, 1 minute at 60° C.

The experiment is performed on an ABI Prism 7700 Sequence Detector (PE Applied Biosystems, CA). At the end of the run, fluorescence data acquired during PCR are processed as described in the ABI Prism 7700 user's manual in order to achieve better background subtraction as well as signal linearity with the starting target quantity.

### EXAMPLE 7

Quantitative Expression Profiling of the Novel Human Chemokine Receptor like mRNA

Expression profiling is based on a quantitative polymerase chain reaction (PCR) analysis, also called kinetic analysis, first described in Higuchi et al., 1992 and Higuchi et al., 1993. The principle is that at any given cycle within the exponential phase of PCR, the amount of product is proportional to the initial number of template copies. Using this technique, the expression levels of particular genes, which are transcribed from the chromosomes as messenger RNA (mRNA), are measured by first making a DNA copy (cDNA) of the mRNA, and then performing quantitative PCR on the cDNA, a method called quantitative reverse transcription-polymerase chain reaction (quantitative RT-PCR).

Quantitative RT-PCR analysis of RNA from different human tissues was performed to investigate the tissue distribution of novel C-C chemokine receptor-like mRNA. In most cases, 25 μg of total RNA from various tissues (including Human Total RNA Panel I-V, Clontech Laboratories, Palo Alto, Calif., USA) was used as a template to synthsize first-strand cDNA using the SUPERSCRIP™ First-Strand Synthesis System for RT-PCR (Life Technologies, Rockville, Md., USA). First-strand cDNA synthesis was carried out according to the manufacturer's protocol using oligo (dT) to hybridize to the 3′ poly A tails of mRNA and prime the synthesis reaction. Approximately 10 ng of the first-strand cDNA was then used as template in a polymerase chain reaction. In other cases, 10 ng of commercially available cDNAs (Human Immune System MTC Panel and Human Blood Fractions MTC Panel, Clontech Laboratories, Palo Alto, Calif., USA) were used as template in a polymerase chain reaction. The polymerase chain reaction was performed in a LightCycler (Roche Molecular Biochemicals, Indianapolis, Ind., USA), in the presence of the DNA-binding fluorescent dye SYBR Green I which binds to the minor groove of the DNA double helix, produced only when double-stranded DNA is successfully synthesized in the reaction (Morrison et al., 1998). Upon binding to double-stranded DNA, SYBR Green I emits light that can be quantitatively measured by the LightCycler machine. The polymerase chain reaction was carried out using oligonucleotide primers LBRI_{—}263_DNA-L1 (SEQ ID NO: 10,) and LBRI_{—}263_DNA-R2 (SEQ ID NO: 11) and measurements of the intensity of emitted light were taken following each cycle of the reaction when the reaction had reached a temperature of 81 degrees C. Intensities of emitted light were converted into copy numbers of the gene transcript per nanogram of template cDNA by comparison with simultaneously reacted standards of known concentration.

To correct for differences in mRNA transcription levels per cell in the various tissue types, a normalization procedure was performed using similarly calculated expression levels in the various tissues of five different housekeeping genes: glyceraldehyde-3-phosphatase (G3PDH), hypoxanthine guanine phophoribosyl transferase (HPRT), beta-actin, porphobilinogen deaminase (PBGD), and beta-2-microglobulin The level of housekeeping gene expression is considered to be relatively constant for all tissues (Adams et al., 1993, Adams et al., 1995, Liew et al., 1994) and therefore can be used as a gauge to approximate relative numbers of cells per .mu.g of total RNA used in the cDNA synthesis step. Except for the use of a slightly different set of housekeeping genes and the use of the LightCycler system to measure expression levels, the normalization procedure was similar to that described in the RNA Master Blot User Manual, Apendix C (1997, Clontech Laboratories, Palo Alto, Calif., USA). In brief, expression levels of the five housekeeping genes in all tissue samples were measured in three independent reactions per gene using the LightCycler and a constant amount (25 .mu.g) of starting RNA. The calculated copy numbers for each gene, derived from comparison with simultaneously reacted standards of known concentrations, were recorded and the mean number of copies of each gene in all tissue samples was determined. Then for each tissue sample, the expression of each housekeeping gene relative to the mean was calculated, and the average of these values over the five housekeeping genes was found. A normalization factor for each tissue was then calculated by dividing the final value for one of the tissues arbitrarily selected as a standard by the corresponding value for each of the tissues. To normalize an experimentally obtained value for the expression of a particular gene in a tissue sample, the obtained value was multiplied by the normalization factor for the tissue tested. This normalization method was used for all tissues except those derived from the Human Blood Fractions MTC Panel, which showed dramatic variation in some housekeeping genes depending on whether the tissue had been activated or not. In these tissues, normalization was carried out with a single housekeeping gene, beta-2-microglobulin.

Results are shown in FIGS. 13 and 14, showing the experimentally obtained copy numbers of mRNA per 10 ng of first-strand cDNA on the left and the normalized values on the right. RNAs used for the cDNA synthesis, along with their supplier and catalog numbers are shown in tables 1 and 2.

References

Higuchi, R., Dollinger, G., Walsh, P. S. and Griffith, R. (1992) Simultaneous amplification and detection of specific DNA sequences. BioTechnology 10:413-417.

Higuchi, R, Fockler, C., Dollinger, G. and Watson, R. (1993) Kinetic PCR analysis: real-time monitoring of DNA amplification reactions. BioTechnology 11:1026-1030.

T. B. Morrison, J. J. Weis & C. T. Wittwer .(1998) Quantification of low-copy transcripts by continuous SYBR Green I monitoring during amplification. Biotechniques 24:954-962.

Adams, M. D., Kerlavage, A. R., Fields, C. & Venter, C. (1993) 3,400 new expressed sequence tags identify diversity of transcripts in human brain. Nature Genet. 4:256-265.

Adams, M. D., et al. (1995) Initial assessment of human gene diversity and expression patterns based upon 83 million nucleotides of cDNA sequence. Nature 377 supp:3-174.

Liew, C. C., Hwang, D. M., Fung, Y. W., Laurenson, C., Cukerman, E., Tsui, S. & Lee, C. Y. (1994) A catalog of genes in the cardiovascular system as identified by expressed sequence tags. Proc. Natl. Acad. Sci. USA 91:10145-10649.

TABLE 1
Whole-body-screen tissues

| Tissue | Supplier | Panel name and catalog number |
|---|---|---|
| 1. brain | Clontech | Human Total RNA Panel I, K4000-1 |
| 2. heart | Clontech | Human Total RNA Panel I, K4000-1 |
| 3. kidney | Clontech | Human Total RNA Panel I, K4000-1 |
| 4. liver | Clontech | Human Total RNA Panel I, K4000-1 |
| 5. lung | Clontech | Human Total RNA Panel I, K4000-1 |
| 6. trachea | Clontech | Human Total RNA Panel I, K4000-1 |
| 7. bone marrow | Clontech | Human Total RNA Panel II, K4001-1 |
| 8. colon | Clontech | Human Total RNA Panel II, K4001-1 |
| 9. small intestine | Clontech | Human Total RNA Panel II, K4001-1 |
| 10. spleen | Clontech | Human Total RNA Panel II, K4001-1 |
| 11. stomach | Clontech | Human Total RNA Panel II, K4001-1 |
| 12. thymus | Clontech | Human Total RNA Panel II, K4001-1 |
| 13. mammary gland | Clontech | Human Total RNA Panel III, K4002-1 |
| 14. skeletal muscle | Clontech | Human Total RNA Panel III, K4002-1 |
| 15. prostate | Clontech | Human Total RNA Panel III, K4002-1 |
| 16. testis | Clontech | Human Total RNA Panel III, K4002-1 |
| 17. uterus | Clontech | Human Total RNA Panel III, K4002-1 |
| 18. cerebellum | Clontech | Human Total RNA Panel IV, K4003-1 |
| 19. fetal brain | Clontech | Human Total RNA Panel IV, K4003-1 |
| 20. fetal liver | Clontech | Human Total RNA Panel IV, K4003-1 |
| 21. spinal cord | Clontech | Human Total RNA Panel IV, K4003-1 |
| 22. placenta | Clontech | Human Total RNA Panel IV, K4003-1 |
| 23. adrenal gland | Clontech | Human Total RNA Panel V, K4004-1 |
| 24. pancreas | Clontech | Human Total RNA Panel V, K4004-1 |
| 25. salivary gland | Clontech | Human Total RNA Panel V, K4004-1 |
| 26. thyroid | Clontech | Human Total RNA Panel V, K4004-1 |

TABLE 2
Blood/lung-screen tissues

| Tissue | Supplier | Panel name and catalog number |
|---|---|---|
| 1. lymph node | Clontech | Human Immune System MTC Panel, |
| | | K1426-1 |
| 2. peripheral blood | Clontech | Human Immune System MTC Panel, |
| leukocytes | | K1426-1 |
| 3. tonsil | Clontech | Human Immune System MTC Panel, |
| | | K1426-1 |
| 4. peripheral blood | Clontech | Human Blood Fractions MTC Panel, |
| mononuclear cells | | K1426-1 |
| 5. peripheral | Clontech | Human Blood Fractions MTC Panel, |
| bloodmononuclear | | K1426-1 |
| cells - | | |
| activated | | |
| 6. T-cell (CD8+) | Clontech | Human Blood Fractions MTC Panel, |
| | | K1426-1 |
| 7. T-cell (CD8+) - | Clontech | Human Blood Fractions MTC Panel, |
| activated | | K1426-1 |
| 8. T-cell (CD4+) | Clontech | Human Blood Fractions MTC Panel, |
| | | K1426-1 |
| 9. T-cell (CD4+) - | Clontech | Human Blood Fractions MTC Panel, |
| activated | | K1426-1 |
| 10. B-cell (CD19+) | Clontech | Human Blood Fractions MTC Panel, |
| | | K1426-1 |
| 11. B-cell (CD19+) - | Clontech | Human Blood Fractions MTC Panel, |
| activated | | K1426-1 |
| 12. Monocytes | Clontech | Human Blood Fractions MTC Panel, |
| (CD14+) | | K1426-1 |
| 13. Th1 clone | In-house | |
| 14. Th2 clone | In-house | |
| 15. neutrophil | In-house | |
| 16. neutrophil | In-house | |
| 17. Normal Bronchial/ | In-house | |
| Tracheal | | |
| Epithelial | | |
| Cells | | |
| 18. Normal Bronchial/ | In-house | |
| Tracheal smooth | | |
| muscle cell | | |
| 19. Normal lung | In-house | |
| fibroblast | | |
| 20. Microvascular | In-house | |
| Endothelial | | |
| cell | | |
| 21. U937 | In-house | |
| 22. RAMOS | In-house | |
| 23. Jurkat | In-house | |
| 24. HelaS3 | In-house | |
| 25. IMR-90 | In-house | |
| 26. HEK293 | In-house | |

### EXAMPLE 8

Treatment of a Patient with a Reagent which Specifically Binds to an Human Chemokine receptor-like mRNA

Synthesis of an antisense oligonucleotide comprising at least 11 contiguous nucleotides selected from the complement of SEQ ID NO: 1 is performed on a Pharmacia Gene Assembler series synthesizer using the phosphoramidite procedure (Uhlmann et al., Chem. Rev. 90, 534-83, 1990). Following assembly and deprotection, the oligonucleotide is twice ethanol-precipitated, dried, and suspended in phosphate-buffered saline (PBS) at the desired concentration. Purity of the oligonucleotide is tested by capillary gel electrophoreses and ion exchange HPLC. The endotoxin level in the oligonucleotide preparation is determined using the Limulus Amebocyte Assay (Bang, Biol. Bull. (Woods Hole, Mass) 105, 361-362, 1953).

An aqueous composition containing the antisense oligonucleotides at a concentration of 0.1-100 μM is administered directly to a patient having by injection. The severity of the patient is decreased.

### EXAMPLE 9

In vivo Testing of Compounds/target Validation for Asthma Treatment

1. Tests for Activity of T Cells

Costimulatory molecules-cytokines, cytokine receptors, signalling molecules, any molecule involved in T cell activation

Mouse anti-CD3 induced cytokine production model

BALB/c mice were injected with a single intravenous injection of 10 μg of 145-2C11 (purified hamster anti-mouse CD3 ε monoclonal antibodies, PHARMINGEN). Compound was administered intraperitoneally 60 min prior to the anti-CD3 mAb injection. Blood was collected 90 min after the antibody injection. Serum was obtained by centrifugation at 3000 r.p.m. for 10 min. IL-2 and IL-4 levels in the serum was determined by an ELISA.

2. Tests for Activity of B Cells

B cell receptor, signalling molecules, any molecule involved in B cell activation/Ig class switching

Mouse anti-IgD induced IgE production model

BALB/c mice were injected intravenously with 0.8 mg of purified goat anti-mouse IgD antibody or PBS (defined as day 0). Compound was administered intraperitoneally from day 0 to day 6. On day 7 blood was collected and serum was obtained by centrifugation at 3000 r.p.m. for 10 min. Serum total levels of IgE were determined by YAMASA's ELISA kit and their Ig subtypes were done by an Ig ELISA KIT (Rougier Bio-tech's, Montreal, Canada).

3. Tests for Activity of Monocytes/macrophages, Signalling Molecules, Transcription Factors

Mouse LPS-induced TNF-α Production Model

BALB/c mice were injected intraperitoneally with LPS (200 μg/mouse). Compound was administered intraperitoneally 1 hr before the LPS injection. Blood was collected at 90 min post-LPS injection and plasma was obtained. TNF-α concentration in the sample was determined using an ELISA kit.

4. Tests Eosinophil Activation

Eotaxin-eotaxin receptor (GPCR) Signalling molecules, Cytoskeletal molecules, adhision molecules Mouse eotaxin-induced eosinophilia model

BALB/c mice were injected intradermally with a 2.5 ml of air on days −6 and −3 to prepare airpouch. On day 0 compound was administered intraperitoneally 60 min before eotaxin injection (3 μg/mouse, i.d.). IL-5 (300 ng/mouse) was injected intravenously 30 min before the eotaxin injection. After 4 hr of the eotaxin injection leukocytes in exudate was collected and the number of total cells was counted. The differential cell counts in the exudate were performed by staining with May-Grunwald Gimsa solution.

5. Tests Activation of Th2 Cells

Molecules involved in antigen presentation, costimulatory molecules, signaling molecules, transcription factors

Mouse D10 cell transfer model

D10.G4.1 cells (1×107 cells/mouse) containing 2 mg of conalbumin in saline was administered i.v. to AKR mice. After 6 hr blood was collected and serum was obtained by centifugation at 3000 r.p.m. for 10 min. IL-4 and IL-5 level in serum were determined by ELISA kits. Compound was admimintered intraperitoneally at −4 and +1 hr after these cells injection.

6. Passive Cutaneous Anaphylaxis (PCA) Test in Rats

6 Weeks old male Wistar rats are sensitized intradermally (i.d.) on their shaved backs with 50 μl of 0.1 μg/ml mouse anti-DNP IgE monoclonal antibody (SPE-7) under a light anesthesia. After 24 hours, the rats are challenged intravenously with 1 ml of saline containing 0.6 mg DNP-BSA (30) (LSL CO., LTD) and 0.005 g of Evans blue. Compounds are injected intraperitoneally (i.p.) 0.5 hr prior to antigen injection. Rats without the sensitization, challenge, and compound treatment are used for a blank (control) and rats with sensitization, challenge and vehicle treatment are used to determine a value without inhibition. Thirty min after the challenge, the rats are killed, and the skin of the back is removed. Evans blue dye in the skin is extracted in formamide overnight at 63° C. Then an absorbance at 620 nm is measured to obtain the optical density of the leaked dye.

Percent inhibition of PCA with a compound is calculated as follows: % inhibition={(mean vehicle value—sample value)/(mean vehicle value—mean control value)}×100

7. Anaphylactic Bronchoconstriction in Rats

6 Weeks old male Wistar rats are sensitized intravenously (i.v.) with 10 μg mouse anti-DNP IgE, SPE-7, and 1 days later, the rats are challenged intravenously with 0.3 ml of saline containing 1.5 mg DNP-BSA (30) under anesthesia with urethan (1000 mg/kg, i.p.) and gallamine (50 mg/kg, i.v.). The trachea is cannulated for artifical respiration (2 ml/stroke, 70 strokes/min). Pulmonary inflation pressure (PIP) is recorded thruogh a side-arm of cannula connected to pressure transducer. Change in PIP reflects change of both resistance and compliance of the lungs. To evaluate the drugs, each drug is given i.v. 5 min before challenge.

1. An isolated polynucleotide encoding a chemokine-like receptor polypeptide and being selected from the group consisting of:
a) a polynucleotide encoding a chemokine-like receptor polypeptide comprising an amino acid sequence selected form the group consisting of:
amino acid sequences which are at least about 26% identical to the amino acid sequence shown in SEQ ID NO: 2; the amino acid sequence shown in SEQ ID NO: 2;
amino acid sequences which are at least about 26% identical to the amino acid sequence shown in SEQ ID NO: 7; the amino acid sequence shown in SEQ ID NO: 7;
amino acid sequences which are at least about 26% identical to the amino acid sequence shown in SEQ ID NO: 8; and the amino acid sequence shown in SEQ ID NO: 8.
b) a polynucleotide comprising the sequence of SEQ ID NOS: 1, 4, 5, or 9;
c) a polynucleotide which hybridizes under stringent conditions to a polynucleotide specified in (a) and (b);
d) a polynucleotide the sequence of which deviates from the poly-nucleotide sequences specified in (a) to (c) due to the degeneration of the genetic code; and
e) a polynucleotide which represents a fragment, derivative or allelic variation of a polynucleotide sequence specified in (a to (d).

2. An expression vector containing any polynucleotide of claim 1.

3. A host cell containing the expression vector of claim 2.

4. A substantially purified chemokine-like receptor polypeptide encoded by a polynucleotide of claim 1.

5. A method for producing a chemokine-like receptor polypeptide, wherein the method comprises the following steps:
a) culturing the host cell of claim 3 under conditions suitable for the expression of the chemokine-like receptor polypeptide; and
b) recovering the chemokine-like receptor polypeptide from the host cell culture.

6. A method for detection of a polynucleotide encoding a chemokine-like receptor polypeptide in a biological sample comprising the following steps:
a) hybridizing any polynucleotide of claim 1 to a nucleic acid material of a biological sample, thereby forming a hybridization complex; and
b) detecting said hybridization complex.

7. The method of claim 6, wherein before hybridization, the nucleic acid material of the biological sample is amplified.

8. A method for the detection of a polynucleotide of claim 1 or a chemokine-like receptor polypeptide of claim 4 comprising the steps of:
contacting a biological sample with a reagent which specifically interacts with the polynucleotide or the chemokine-like receptor polypeptide.

9. A diagnostic kit for conducting the method of any one of claims 6 to 8.

10. A method of screening for agents which decrease the activity of a chemokine-like receptor, comprising the steps of:
contacting a test compound with any chemokine-like receptor polypeptide encoded by any polynucleotide of claim 1;
detecting binding of the test compound to the chemokine-like receptor polypeptide, wherein a test compound which binds to the polypeptide is identified as a potential therapeutic agent for decreasing the activity of a chemokine-like receptor.

11. A method of screening for agents which regulate the activity of a chemokine-like receptor, comprising the steps of:
contacting a test compound with a chemokine-like receptor polypeptide encoded by any polynucleotide of claim 1; and
detecting a chemokine-like receptor activity of the polypeptide, wherein a test compound which increases the chemokine-like receptor activity is identified as a potential therapeutic agent for increasing the activity of the chemokine-like receptor, and wherein a test compound which decreases the chemokine-like receptor activity of the polypeptide is identified as a potential therapeutic agent for decreasing the activity of the chemokine-like receptor.

12. A method of screening for agents which decrease the activity of a chemokine-like receptor, comprising the steps of:
contacting a test compound with any polynucleotide of claim 1 and detecting binding of the test compound to the polynucleotide, wherein a test compound which binds to the polynucleotide is identified as a potential therapeutic agent for decreasing the activity of chemokine-like receptor.

13. A method of reducing the activity of chemokine-like receptor, comprising the steps of:
contacting a cell with a reagent which specifically binds to any polynucleotide of claim 1 or any chemokine-like receptor polypeptide of claim 4, whereby the activity of chemokine-like receptor is reduced.

14. A reagent that modulates the activity of a chemokine-like receptor poly-peptide or a polynucleotide wherein said reagent is identified by the method of any of the claim 10 to 12.

15. A pharmaceutical composition, comprising:
the expression vector of claim 2 or the reagent of claim 14 and a pharmaceutically acceptable carrier.

16. Use of the expression vector of claim 2 or the reagent of claim 14 in the preparation of a medicament for modulating the activity of a chemokine-like receptor in a disease.

17. Use of claim 16 wherein the disease is HIV infection, a cardiovascular disorder, asthma or COPD.

18. A cDNA encoding a polypeptide comprising the amino acid sequence shown in SEQ ID NOS: 2, 7 or 8.

19. The cDNA of claim 18 which comprises SEQ ID NOS: 1, 4, 5 or 9.

20. The cDNA of claim 18 which consists of SEQ ID NOS: 1, 4, 5 or 9.

21. An expression vector comprising a polynucleotide which encodes a polypeptide comprising the amino acid sequence shown in SEQ ID NOS: 2, 7 or 8.

22. The expression vector of claim 21 wherein the polynucleotide consists of SEQ ID NOS: 1, 4, 5 or 9.

23. A host cell comprising an expression vector which encodes a polypeptide comprising the amino acid sequence shown in SEQ ID NOS: 2, 7 or 8.

24. The host cell of claim 23 wherein the polynucleotide consists of SEQ ID NOS: 1, 4, 5 or 9.

25. A purified polypeptide comprising the amino acid sequence shown in SEQ ID NOS: 2, 7 or 8.

26. The purified polypeptide of claim 25 which consists of the amino acid sequence shown in SEQ ID NOS: 2, 7 or 8.

27. A fusion protein comprising a polypeptide having the amino acid sequence shown in SEQ ID NOS: 2, 7 or 8.

28. A method of producing a polypeptide comprising the amino acid sequence shown in SEQ ID NOS: 2, 7 or 8, comprising the steps of:
culturing a host cell comprising an expression vector which encodes the polypeptide under conditions whereby the polypeptide is expressed; and
isolating the polypeptide.

29. The method of claim 28 wherein the expression vector comprises SEQ ID NOS: 1, 4, 5 or 9.

30. A method of detecting a coding sequence for a polypeptide comprising the amino acid sequence shown in SEQ ID NO: 2, comprising the steps of:
hybridizing a polynucleotide comprising 11 contiguous nucleotides of SEQ ID NOS: 1, 4, 5 or 9 to nucleic acid material of a biological sample, thereby forming a hybridization complex; and
detecting the hybridization complex.

31. The method of claim 30 further comprising the step of amplifying the nucleic acid material before the step of hybridizing.

32. A kit for detecting a coding sequence for a polypeptide comprising the amino acid sequence shown in SEQ ID NOS: 2, 7 or 8, comprising:
a polynucleotide comprising 11 contiguous nucleotides of SEQ ID NOS: 1, 4, 5 or 9; and
instructions for the method of claim 30.

33. A method of detecting a polypeptide comprising the amino acid sequence shown in SEQ ID NOS: 2, 7 or 8, comprising the steps of:
contacting a biological sample with a reagent that specifically binds to the polypeptide to form a reagent-polypeptide complex; and
detecting the reagent-polypeptide complex.

34. The method of claim 33 wherein the reagent is an antibody.

35. A kit for detecting a polypeptide comprising the amino acid sequence shown in SEQ ID NOS: 2, 7 or 8, comprising:
an antibody which specifically binds to the polypeptide; and
instructions for the method of claim 33.

36. A method of screening for agents which can modulate the activity of a human chemokine-like receptor, comprising the steps of:
contacting a test compound with a polypeptide comprising an amino acid sequence selected from the group consisting of: (1) amino acid sequences which are at least about 26% identical to the amino acid sequence shown in SEQ ID NOS: 2, 7 or 8 and (2) the amino acid sequence shown in SEQ ID NOS: 2, 7 or 8; and
detecting binding of the test compound to the polypeptide, wherein a test compound which binds to the polypeptide is identified as a potential agent for regulating activity of the human chemokine-like receptor.

37. The method of claim 36 wherein the step of contacting is in a cell.

38. The method of claim 36 wherein the cell is in vitro.

39. The method of claim 36 wherein the step of contacting is in a cell-free system.

40. The method of claim 36 wherein the polypeptide comprises a detectable label.

41. The method of claim 36 wherein the test compound comprises a detectable label.

42. The method of claim 36 wherein the test compound displaces a labeled ligand which is bound to the polypeptide.

43. The method of claim 36 wherein the polypeptide is bound to a solid support.

44. The method of claim 36 wherein the test compound is bound to a solid support.

45. A method of screening for agents which modulate an activity of a human chemokine-like receptor, comprising the steps of:
contacting a test compound with a polypeptide comprising an amino acid sequence selected from the group consisting of: (1) amino acid sequences which are at least about 26% identical to the amino acid sequence shown in SEQ ID NOS: 2, 7 or 8 and (2) the amino acid sequence shown in SEQ ID NOS: 2, 7 or 8; and
detecting an activity of the polypeptide, wherein a test compound which increases the activity of the polypeptide is identified as a potential agent for increasing the activity of the human chemokine-like receptor, and wherein a test compound which decreases the activity of the polypeptide is identified as a potential agent for decreasing the activity of the human chemokine-like receptor.

46. The method of claim 45 wherein the step of contacting is in a cell.

47. The method of claim 45 wherein the cell is in vitro.

48. The method of claim 45 wherein the step of contacting is in a cell-free system.

49. A method of screening for agents which modulate an activity of a human chemokine-like receptor, comprising the steps of:
contacting a test compound with a product encoded by a polynucleotide which comprises the nucleotide sequence shown in SEQ ID NOS: 1, 4, 5 or 9; and
detecting binding of the test compound to the product, wherein a test compound which binds to the product is identified as a potential agent for regulating the activity of the human chemokine-like receptor.

50. The method of claim 49 wherein the product is a polypeptide.

51. The method of claim 49 wherein the product is RNA.

52. A method of reducing activity of a human chemokine-like receptor, comprising the step of:
contacting a cell with a reagent which specifically binds to a product encoded by a polynucleotide comprising the nucleotide sequence shown in SEQ ID NOS: 1, 4, 5 or 9, whereby the activity of a human chemokine-like receptor is reduced.

53. The method of claim 52 wherein the product is a polypeptide.

54. The method of claim 53 wherein the reagent is an antibody.

55. The method of claim 52 wherein the product is RNA.

56. The method of claim 55 wherein the reagent is an antisense oligonucleotide.

57. The method of claim 56 wherein the reagent is a ribozyme.

58. The method of claim 52 wherein the cell is in vitro.

59. The method of claim 52 wherein the cell is in vivo.

60. A pharmaceutical composition, comprising:
a reagent which specifically binds to a polypeptide comprising the amino acid sequence shown in SEQ ID NOS: 2, 7 or 8; and
a pharmaceutically acceptable carrier.

61. The pharmaceutical composition of claim 60 wherein the reagent is an antibody.

62. A pharmaceutical composition, comprising:
a reagent which specifically binds to a product of a polynucleotide comprising the nucleotide sequence shown in SEQ ID NOS: 1, 4, 5, or 9; and
a pharmaceutically acceptable carrier.

63. The pharmaceutical composition of claim 62 wherein the reagent is a ribozyme.

64. The pharmaceutical composition of claim 62 wherein the reagent is an antisense oligonucleotide.

65. The pharmaceutical composition of claim 62 wherein the reagent is an antibody.

66. A pharmaceutical composition, comprising:
an expression vector encoding a polypeptide comprising the amino acid sequence shown in SEQ ID NOS: 2, 7 or 8; and
a pharmaceutically acceptable carrier.

67. The pharmaceutical composition of claim 66 wherein the expression vector comprises SEQ ID NOS: 1, 4, 5 or 9.

68. A method of treating a chemokine-like receptor dysfunction related disease, wherein the disease is selected from HIV infection, a cardiovascular disorder, asthma or COPD comprising the step of:
administering to a patient in need thereof a therapeutically effective dose of a reagent that modulates a function of a human chemokine-like receptor, whereby symptoms of the chemokine-like receptor disfunction related disease are ameliorated.

69. The method of claim 68 wherein the reagent is identified by the method of claim 36.

70. The method of claim 68 wherein the reagent is identified by the method of claim 45.

71. The method of claim 68 wherein the reagent is identified by the method of claim 49.


SEQUENCE LISTING (11 sequences)

SEQ ID NO: 1 - DNA, 1059 bp, Homo sapiens
atggagcacacgcacgcccacctcgcagccaacagctcgctgtcttggtggtcccccggctcggcctgcggcttgggtttcgtgcccgtggtctactacagcctcttgctgtgcctcggtttaccagcaaatatcttgacagtgatcatcctctcccagctggtggcaagaagacagaagtcctcctacaactatctcttggcactcgctgctgccgacatcttggtcctctttttcatagtgtttgtggacttcctgttggaagatttcatcttgaacatgcagatgcctcaggtccccgacaagatcatagaagtgctggaattctcatccatccacacctccatatggattactgtaccgttaaccattgacaggtatatcgctgtctgccacccgctcaagtaccacacggtctcatacccagcccgcacccggaaagtcattgtaagtgtttacatcacctgcttcctgaccagcatcccctattactggtggcccaacatctggactgaagactacatcagcacctctgtgcatcacgtcctcatctggatccactgcttcaccgtctacctggtgccctgctccatcttcttcatcttgaactcaatcattgtgtacaagctcaggaggaagagcaattttcgtctccgtggctactccacggggaagaccaccgccatcttgttcaccattacctccatctttgccacactttgggccccccgcatcatcatgattctttaccacctctatggggcgcccatccagaaccgctggctggtacacatcatgtccgacattgccaacatgctagcccttctgaacacagccatcaacttcttcctctactgcttcatcagcaagcggttccgcaccatggcagccgccacgctcaaggctttcttcaagtgccagaagcaacctgtacagttctacaccaatcataacttttccataacaagtagcccctggatctcgccggcaaactcacactgcatcaagatgctggtgtaccagtatgacaaaaatggaaaacctataaaagtatccccg

SEQ ID NO: 2 - protein, 353 aa, Homo sapiens
MEHTHAHLAANSSLSWWSPGSACGLGFVPVVYYSLLLCLGLPANILTVIILSQLVARRQKSSYNYLLALAAADILVLFFIVFVDFLLEDFILNMQMPQVPDKIIEVLEFSSIHTSIWITVPLTIDRYIAVCHPLKYHTVSYPARTRKVIVSVYITCFLTSIPYYWWPNIWTEDYISTSVHHVLIWIHCFTVYLVPCSIFFILNSIIVYKLRRKSNFRLRGYSTGKTTAILFTITSIFATLWAPRIIMILYHLYGAPIQNRWLVHIMSDIANMLALLNTAINFFLYCFISKRFRTMAAATLKAFFKCQKQPVQFYTNHNFSITSSPWISPANSHCIKMLVYQYDKNGKPIKVSP

SEQ ID NO: 3 - protein, 355 aa, Cercopithecus aethiops
MTTSLYTVETFGPTSYDDDMGLLCEKADVGALIAQFVPPLYSLVFTVGLLGNVVVVMILIKYRRLRIMTNIYLLNLAISDLLFLFTLPFWIHYVREHNWVFSHGMCKVLSGFYHTGLYSEIFFIILLTIDRYLAIVHAVFALRARTVTFGVITSIVTWGLAVLVALPEFIFYGTEELFPETLCSAIYPQDTVYSWRHFHTLKMTILCLALPLLVMAICYTGIIKTLLKCPSKKKYKAIRLIFVIMAVFFIFWTPYNVAILISTYQSILFGLDCERSKHVDLVVLVTEVIAYSHCCVNPVIYAFVGERFRKYLRHFFHRHVLMHLGRYIPFLPSEKLERTSSVSPSTAEPELCIVF

SEQ ID NO: 4 - DNA, 1070 bp, Homo sapiens
atgtatctgagaacttaggaccaccctggtgcatcaagatgcttccactcaaagaagttcatggaagtcgtctgactgagggacagatctcccatctcccacgctccccagggcgtatgctcattgagtggaatgcaaatatcttgacagtgatcatcctctcccagctggtggcaagaagacagaagtcctcctacaactatctcttggcactcgctgctgccgacatcttggtcctctttttcatagtgtttgtggacttcctgttggaagatttcatcttgaacatgcagatgcctcaggtccccgacaagatcatagaagtgctggaattctcatccatccacacctccatatggattactgtaccgttaaccattgacaggtatatcgctgtctgccacccgctcaagtaccacacggtctcatacccagcccgcacccggaaagtcattgtaagtgtttacatcacctgcttcctgaccagcatcccctattactggtggcccaacatctggactgaagactacatcagcacctctgtgcatcacgtcctcatctggatccactgcttcaccgtctacctggtgccctgctccatcttcttcatcttgaactcaatcattgtgtacaagctcaggaggaagagcaattttcgtctccgtggctactccacggggaagaccaccgccatcttgttcaccattacctccatctttgccacactttgggccccccgcatcatcatgattctttaccacctctatggggcgcccatccagaaccgctggctggtgcacatcatgtccgacattgccaacatgctagcccttctgaacacagccatcaacttcttcctctactgcttcatcagcaagcggttccgcaccatggcagccgccacgctcaaggctttcttcaagtgccagaagcaacctgtacagttctacaccaatcataacttttccataacaagtagcccctggatctcgccggcaaactcacactgcatcaagatgctggtgtaccagtatgacaaaaatggaaaacctataaaagtatccccgtga

SEQ ID NO: 5 - DNA, 1032 bp, Homo sapiens
atgcttccactcaaagaagttcatggaagtcgtctgactgagggacagatctcccatctcccacgctccccagggcgtatgctcattgagtggaatgcaaatatcttgacagtgatcatcctctcccagctggtggcaagaagacagaagtcctcctacaactatctcttggcactcgctgctgccgacatcttggtcctctttttcatagtgtttgtggacttcctgttggaagatttcatcttgaacatgcagatgcctcaggtccccgacaagatcatagaagtgctggaattctcatccatccacacctccatatggattactgtaccgttaaccattgacaggtatatcgctgtctgccacccgctcaagtaccacacggtctcatacccagcccgcacccggaaagtcattgtaagtgtttacatcacctgcttcctgaccagcatcccctattactggtggcccaacatctggactgaagactacatcagcacctctgtgcatcacgtcctcatctggatccactgcttcaccgtctacctggtgccctgctccatcttcttcatcttgaactcaatcattgtgtacaagctcaggaggaagagcaattttcgtctccgtggctactccacggggaagaccaccgccatcttgttcaccattacctccatctttgccacactttgggccccccgcatcatcatgattctttaccacctctatggggcgcccatccagaaccgctggctggtgcacatcatgtccgacattgccaacatgctagcccttctgaacacagccatcaacttcttcctctactgcttcatcagcaagcggttccgcaccatggcagccgccacgctcaaggctttcttcaagtgccagaagcaacctgtacagttctacaccaatcataacttttccataacaagtagcccctggatctcgccggcaaactcacactgcatcaagatgctggtgtaccagtatgacaaaaatggaaaacctataaaagtatccccgtga

SEQ ID NO: 6 - DNA, 1826 bp, Homo sapiens
tggctctcatttagggaccatattgtgtgattctaatgtatctgagaacttaggaccaccctggtgcatcaagatgcttccactcaaagaagttcatggaagtcgtctgactgagggacagatctcccatctcccacgctccccagggcgtatgctcattgagtggaatgcaaatatcttgacagtgatcatcctctcccagctggtggcaagaagacagaagtcctcctacaactatctcttggcactcgctgctgccgacatcttggtcctctttttcatagtgtttgtggacttcctgttggaagatttcatcttgaacatgcagatgcctcaggtccccgacaagatcatagaagtgctggaattctcatccatccacacctccatatggattactgtaccgttaaccattgacaggtatatcgctgtctgccacccgctcaagtaccacacggtctcatacccagcccgcacccggaaagtcattgtaagtgtttacatcacctgcttcctgaccagcatcccctattactggtggcccaacatctggactgaagactacatcagcacctctgtgcatcacgtcctcatctggatccactgcttcaccgtctacctggtgccctgctccatcttcttcatcttgaactcaatcattgtgtacaagctcaggaggaagagcaattttcgtctccgtggctactccacggggaagaccaccgccatcttgttcaccattacctccatctttgccacactttgggccccccgcatcatcatgattctttaccacctctatggggcgcccatccagaaccgctggctggtgcacatcatgtccgacattgccaacatgctagcccttctgaacacagccatcaacttcttcctctactgcttcatcagcaagcggttccgcaccatggcagccgccacgctcaaggctttcttcaagtgccagaagcaacctgtacagttctacaccaatcataacttttccataacaagtagcccctggatctcgccggcaaactcacactgcatcaagatgctggtgtaccagtatgacaaaaatggaaaacctataaaagtatccccgtgattccataggtgtggcaactactgcctctgtctaatccatttccagatgggaaggtgtcccatcctatggctgagcagctctccttaagagtgctaatccgatttcctgtctcccgcagactgggcaattctcagactggtagatgagaagagatggaagagaagaaaggagagcatgaagcttgtttttacttatgcatttatttccacagagtcgtaatgacagcaaaagctcctaccagtttgaagatgccattggagcttgtgtcatcatcctgtgaccagttaggacacaaagtagagaagtagtctgtgatttcgccctggtaccatccacagtcactgggaacccttcatttatgggacttaccaagccccagtagcacatagctgagcctgcactcttcttccgagagctgaggtcattcatcacttccctctgctgttcccaggagctaacaataatgactatttcaggatttttttcaaggtgccctttgtcctagagagggttgtggtcttgaattggctctggcactcctagcttcagaatgacactgtgggaatagaagagtattggatcccatccaaactgtggccagagcttcttcaggaaatctccaaacccgcatagctgtgacctcaaacctggggtctaaaaggcagttttctatttatcattatgtatagattttctctatctcctccaaaacaaagaccct

SEQ ID NO: 7 - protein, 356 aa, Homo sapiens
MYLRTLGPPWCIKMLPLKEVHGSRLTEGQISHLPRSPGRMLIEWNANILTVIILSQLVARRQKSSYNYLLALAAADILVLFFIVFVDFLLEDFILNMQMPQVPDKIIEVLEFSSIHTSIWITVPLTIDRYIAVCHPLKYHTVSYPARTRKVIVSVYITCFLTSIPYYWWPNIWTEDYISTSVHHVLIWIHCFTVYLVPCSIFFILNSIIVYKLRRKSNFRLRGYSTGKTTAILFTITSIFATLWAPRIIMILYHLYGAPIQNRWLVHIMSDIANMLALLNTAINFFLYCFISKRFRTMAAATLKAFFKCQKQPVQFYTNHNFSITSSPWISPANSHCIKMLVYQYDKNGKPIKVSP

SEQ ID NO: 8 - protein, 343 aa, Homo sapiens
MLPLKEVHGSRLTEGQISHLPRSPGRMLIEWNANILTVIILSQLVARRQKSSYNYLLALAAADILVLFFIVFVDFLLEDFILNMQMPQVPDKIIEVLEFSSIHTSIWITVPLTIDRYIAVCHPLKYHTVSYPARTRKVIVSVYITCFLTSIPYYWWPNIWTEDYISTSVHHVLIWIHCFTVYLVPCSIFFILNSIIVYKLRRKSNFRLRGYSTGKTTAILFTITSIFATLWAPRIIMILYHLYGAPIQNRWLVHIMSDIANMLALLNTAINFFLYCFISKRFRTMAAATLKAFFKCQKQPVQFYTNHNFSITSSPWISPANSHCIKMLVYQYDKNGKPIKVSP

SEQ ID NO: 9 - DNA, 1062 bp, Homo sapiens
atggagcacacgcacgcccacctcgcagccaacagctcgctgtcttggtggtcccccggctcggcctgcggcttgggtttcgtgcccgtggtctactacagcctcttgctgtgcctcggtttaccagcaaatatcttgacagtgatcatcctctcccagctggtggcaagaagacagaagtcctcctacaactatctcttggcactcgctgctgccgacatcttggtcctctttttcatagtgtttgtggacttcctgttggaagatttcatcttgaacatgcagatgcctcaggtccccgacaagatcatagaagtgctggaattctcatccatccacacctccatatggattactgtaccgttaaccattgacaggtatatcgctgtctgccacccgctcaagtaccacacggtctcatacccagcccgcacctattactggtggcccaacatctggactgaagactacatcagcacctctgcccggaaagtcattgtaagtgtttacatcacctgcttcctgaccagcatcctgcatcacgtcctcatctggatccactgcttcaccgtctacctggtgccctgctccatcttcttcatcttgaactcaatcattgtgtacaagctcaggaggaagagcaattttcgtctccgtggctactccacggggaagaccaccgccatcttgttcaccattacctccatctttgccacactttgggccccccgcatcatcatgattctttaccacctctatggggcgcccatccagaaccgctggctggtacacatcatgtccgacattgccaacatgctagcccttctgaacacagccatcaacttcttcctctactgcttcatcagcaagcggttccgcaccatggcagccgccacgctcaaggctttcttcaagtgccagaagcaacctgtacagttctacaccaatcataacttttccataacaagtagcccctggatctcgccggcaaactcacactgcatcaagatgctggtgtaccagtatgacaaaaatggaaaacctataaaagtatccccgtga

SEQ ID NO: 10 - DNA, 24 bp, Homo sapiens
  misc_feature: Primer LBRI_263_DNA-L1
ctgctgccgacatcttggtcctct

SEQ ID NO: 11 - DNA, 24 bp, Homo sapiens
  misc_feature: Primer LBRI_263_DNA-R2
ggtacttgagcgggtggcagacag